(19) United States
(12) Patent Application Publication
(10) Pub. No.: US 2018/0282167 A1
(43) Pub. Date: Oct. 4, 2018

(54) Mechanochemical Processing of Thermoplastic Nanocomposites for Regenerative Orthopedic Surgery

(71) Applicant: Temple University-Of The Commonwealth System of Higher Education, Philadelphia, PA (US)

(72) Inventors: Sean M. Devlin, Philadelphia, PA (US); Peter I. Lelkes, Cherry Hill, NJ (US)

(21) Appl. No.: 15/765,108

(22) PCT Filed: Sep. 30, 2016
(86) PCT No.: PCT/US16/54927

§ 371 Date: Mar. 30, 2018

Related U.S. Application Data
(60) Provisional application No. 62/235,801, filed on Oct. 1, 2015.

(51) Int. Cl. C01B 32/25; A61B 17/86; C08G 63/08; C08K 3/04

(52) U.S. Cl. CPC C01B 32/25; A61B 17/866; A61L 31/125; C08K 3/04; C08G 63/08

(57) ABSTRACT

Described herein are improved surgical fixation devices and methods of making the same. The methods comprise mechanochemical processing and vacuum annealing of biocompatible polymer-nanomaterial mixtures to form composites exhibiting superior mechanical properties.

### CROSS-REFERENCE TO RELATED APPLICATIONS

This application claims priority to U.S. Provisional Patent Application No. 62/235,801 filed Oct. 1, 2015, the contents of which are incorporated by reference herein in their entirety.

### BACKGROUND OF THE INVENTION

Orthopedic fixation devices, such as plates, screws, pins, rods, anchors, and staples are commonly used in a variety of orthopedic procedures, including joint repair, bone grafting, and bone fracture fixation.

The biomechanical properties of the fixation devices often influence the success of the orthopedic procedure. Unfortunately, current degradable orthopedic fixation device materials, such as various polylactides and/or glycolides and their calcium phosphate containing composites, undergo brittle failure and frequently crack during implantation. While the use of biocompatible composites in the manufacture of fixation devices has been explored, calcium phosphate composites are not able to create covalent bonds of the surrounding matrix.

Thus, there is a need in the art for improved orthopedic fixation devices. The present invention satisfies this unmet need.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts the results of experiments investigating the effects of condensation reactions on the mechanical properties of PDLG and PDLG-nanomaterial composites. Rheometry was used to analyze the zero-shear viscosity of the indicated groups before and after vacuum annealing.

FIG. 2 depicts the results of thermo-gravimetric analysis of nanodiamonds before and after sintering.

FIG. 3 depicts the results of thermo-gravimetric analysis of cyromilled PDLG and cyromilled PDLG+1% nanodiamond.

FIG. 4 depicts the results of experiments investigating the effects of condensation reactions on the mechanical properties of PDLG and PDLG-nanomaterial composites. Flexural testing was used to analyze the stress-strain behavior of the indicated groups before and after vacuum annealing.

FIG. 5 is a graph which depicts the flexural modulus of the indicated groups, as quantified from the stress-strain curves of FIG. 4. It was observed that stiffness is increased after vacuum annealing in composites comprising 0.5% nanodiamond (ND).

FIG. 6 is a graph which depicts the ultimate stress of the indicated groups, as quantified from the stress-strain curves of FIG. 4. It was observed that ultimate stress is increased after vacuum annealing in composites comprising 0.5% nanodiamond (ND).

FIG. 7 is a graph which depicts the elongation at break of the indicated groups, as quantified from the stress-strain curves of FIG. 4. It was observed that elongation at break is increased after vacuum annealing in composites comprising nanomaterials.

FIG. 8 is a graph which depicts the toughness of the indicated groups, as quantified from the stress-strain curves of FIG. 4. It was observed that toughness is increased after vacuum annealing in composites comprising nanomaterials.

FIG. 9 is a set of graphs depicting the results of example experiments investigating the flexural stress-strain relationship of various PDLG-nanomaterial composites. PDLG8531 was cryomilled with 0, 0.1, or 0.2% NanoDiamond(ND) and/or HydroxyApatite(HA), n=1. Samples were milled and molded directly without drying under vacuum. Only one sample was molded without milling (black line). Nanodiamonds used in this experiment were UD90 (Nanoblox, Inc.), air oxidized at 450° Celsius for 5 hours.

FIG. 10A and FIG. 10B are a set of graphs depicting the results of example experiments investigating the flexural stress-strain relationship of PDLG biomaterials, alone or together with functionalized nanodiamonds (ND). Materials were neither vacuum dried nor annealed (top row, FIG. 10A and FIG. 10B), vacuum dried at room temperature (middle row, FIG. 10A and FIG. 10B), or vacuum dried and subsequently vacuum annealed above their melt temperature (bottom row, FIG. 10A and FIG. 10B). The columns represent symbols that (1) polymer granules or compression molded has arrived, (2) cryo-milled in a SPEX sample prep, and were cryomilled with 0.1% nanodiamonds enriched with the surface functionalizations of (3) hydroxyl, (4) carboxylic acid, and (5) amine. The results of the first row indicate that vacuum drying at room temperature is necessary to remove residual moisture from the milling process. Nanodiamonds functionalized with hydroxyl groups demonstrate the largest effect on the polymer matrix. Before annealing, the composites are greatly embrittled; subsequent annealing both stiffens and toughens this particular composite combination.

FIG. 11 is a set of graphs depicting the rheometry results of experiments the 5 types of samples from FIG. 10A and FIG. 10B, all of which were vacuum annealed above melting temperature for 72 hours (150° Celsius & 0.2 Torr). Native samples were polymer granules just annealed directly, CM (Cryomilled) samples were milled in the SPEX sample prep, and the OH/COOH/NH2 samples were cryomilled with 0.1% of functionalized nanodiamond. Two millimeter, 25 millimeter diameter thick disk shaped samples were cut from vacuum oven melt annealed samples. The first row represents apparent viscosity as a function of oscillatory frequency. Subsequent rows are derived from this first row: phase angle and the tangent of the phase angle.

FIG. 12 is a set of graphs depicting stress-strain curves produced from sample beams wafered from compression molded disks of the polymer and composites in three-point bend, load to failure. The first graph (left) represent polymer granules that were processed in “as-arrived” condition, only dried under vacuum at room temperature before compression molding. All other samples were annealed above melt temperature under vacuum. Colored lines in these grafts represent groups that were placed in various sections of the vacuum oven to investigate possible temperature variations, from insulated back to uninsulated front glass door: red, magenta, black, cyan, blue.

FIG. 13 is set of graphs derived from the raw data in FIG. 12. The graphs are comparing processing procedure steps as they effect the mechanics of the final material product. Each subplot represents the change in: Ultimate Strain (top left), Ultimate stress (top right), flexural modulus (bottom left) and Yield strength at 0.2% strain. Three (3) groups are depicted in each subplot: (Left) Material as arrived from manufacturing dried under high vacuum overnight at room temperature before compression molding, (Center) Vacuum annealed @ 150 Celsius & 0.2 Torr for 72 hours, & (Right) Cryomilled and Vacuum annealed. Vacuum melt annealing alone both toughens and stiffens the material. Melt annealing under vacuum significantly increases the flexural modulus (p<0.05), even without the addition of nanodiamonds.

FIG. 14 is a set of graphs comparing the mechanical of final material product formed from cryomilling Poly(D,L-lactide-co-glycolide) with surface functionalized detonation nanodiamonds at concentrations of 0%, 0.1%, 0.2%, and 0.5%. The most significant increase ultimate stress and ultimate strain was observed in hydroxyl functionalized nanodiamonds at 0.1% weight blend, wherein further increasing concentration decreases improvements. Yield strength was unaffected by all groups, and only carboxylic acid functionalized nanodiamonds decreased the flexural modulus.

FIG. 15 depicts FTIR-ATR Transmission peaks after normalization and Savitsky-Golay smoothing. Reference peaks are added to highlight areas of interest.

FIG. 16 is a set of images depicting cryomilled PDLG8531 with 7F2 osteoblasts after 3 days in culture.

FIG. 17 is a set of images depicting cryomilled PDLG8531-amine functionalized ND composites with 7F2 osteoblasts after 3 days in culture.

FIG. 18 is an image depicting cryomilled PDLG8531-amine functionalized ND composites without 7F2 osteoblasts after 3 days in culture.

FIG. 19 is a set of graphs depicting the results of experiments investigating cell number and volume cultured on PDLG and PDLG-ND composites. Top: Hydroxyl surface seems to support the most cells per scaffold (n=3). Bottom: Cell size(volume) does not seem to significantly vary between surface groups. Hydroxyl functionalized nanodiamonds also seem to increase the number of cells attached to the biomaterial.

FIG. 20 depicts the setup and results of mechanical testing. (Left) 3-point bend flexural fixture on Bose Electroforce, sample dimensions are 6 mm high and 2 mm deep. Gap distance between lower stanchions is 20 mm. Constant displacement rate (1 mm/minute) until failure. (Right) Stress-Strain curves of the three types of nanodiamond composites (0.1% ND by weight, n=3). The top row was cryomilled (CM) and vacuum dried at room temperature before compression molding. The bottom row was additionally vacuum annealed for SSPC (150° C. at 0.2 Torr for 48 hours). All sets were then compression molded and wafered identically for mechanical testing.

FIG. 21 depicts the results of experiments demonstrating the dynamic rheometry of native polylactide, polylactide that has been CM, and polylactide that has been CM with functional nanodiamonds. All nanodiamonds are 0.1% by weight (n=3).

FIG. 22 depicts the results of flexural testing, constant displacement until failure. Strain at failure displaced as a function of ND-OH weight percentage. N=3 *** p<0.005. Control has undergone CM and SSPC (neither process significantly affects polylactide alone).

FIG. 23 depicts the results of experiments investigating fatigue testing, 1 Hz sinusoidal oscillations to 80 MPa peak flexural stress. Results displayed as number of cycles to failure, for increasing number of processing additions from left to right. Control represents virgin polymer granules dried under vacuum at room temperature before compression molding, vacuum annealing (VA)=SSPC for virgin polymer pellets. VA+CM does not include nanodiamonds, and VA+CM+ND-OH is 0.1% weight. n=3, ** p<0.01.

FIG. 24 depicts bright field microscopy images of 50 μm thick wafers of polylactide and the various nanodiamond composites.

FIG. 25 depicts polarized light microscopy of polylactide (PL) strips after load to failure reveals strain induced birefringence. All nanodiamond shown were used in 0.1% weight percentage. (Left) Virgin granules of polylactide have little ability to distribute load evenly, stress risers are narrow and intense. (Center) ND-COOH composite image is representative of CM and ND-NH2 composites, dark spots with well-defined boundaries are large polylactide granules that did not share in load distribution. (Right) Although still present, the blurring of boundaries around the dark spots is indicative of load sharing.

FIG. 26 depicts the results of experiments comparing differential scanning calorimetry measurements of T_{g} onset for each material group. T_{g} onsets all statistically significant from each other, n=3, p<0.05.

FIG. 27 depicts the results of a degradation study, submerging samples for 9 weeks in cell culture media. All samples were cryomilled and vacuum annealed. n=8. ** p<0.001.

FIG. 28 are pictures of the results of the degradation study, submerging samples for 9 weeks in cell culture media.

FIG. 29 depicts the results of experiments adding 0.1% ND-OH to 50/50 PL/PS blends. Both samples were cryomilled (CM) and Oven Annealed (OA/SSPC), and were compression molded for 7 minutes at 225° C. (Left) No ND-OH, pores are coarse but regular. (Right) 0.1% ND-OH, pore growth has slowed due to viscosity increase, but has not upset viscosity balance of dispersed/matrix phases.

FIG. 30 depicts the results of uniaxial compressive testing of 5 mm tall, 6×6 mm square blocks of porous PL, constant compressive displacement set at 1 mm/minute. Due to irregularities in the heterogeneity of pores as the approach the scale of the block (i.e. mm size), 1 representative curve is depicted from each group based on clustering of characteristics and completeness. n=4.

FIG. 31 depicts the results of experiments investigating surface energy for cross sections of oven annealed, 0.1% ND-OH, and 0.1% HA composites. ND-OH appears to increase wettability of PDLG as much as HA.

FIG. 32 depicts the results of culturing mouse osteoblasts (ATCC 7F2) on cross sectioned wafers of oven annealed, 0.1% ND-OH, and 0.1% HA composites. By 7 days, cells appear confluent on all scaffold types.

FIG. 33 depicts the results of quantifying the cell cultures in FIG. 32 by alamar blue assay. The results confirm comparable numbers to both plain polymer and HA controls by day 10.

### DETAILED DESCRIPTION

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention relates to improved biomaterials with enhanced mechanical properties. In certain embodiments, the biomaterials are used as orthopedic fixation devices, including screws, pins, rods, plates, staples, and the like.

In certain embodiments, the devices of the invention are manufactured by a method which significantly enhances their mechanical properties. The methods described herein are suitable for producing biocompatible and biodegradable fixation devices, which promote the growth of native biological material. Increasing the stiffness, strength, and toughness of orthopedic physician materials would help minimize the amount of material necessary to achieve fixation.

In one aspect, the present invention provides a method of producing degradable biomaterials with increased strength, through the use of the mechanochemical processing of polymer components and nanomaterials to produce a polymer-nanomaterial blend composite. For example, in certain embodiments, solid-state shear pulverization (SSSP) or cryomilling is used to particulate thermoplastic pellets, create reactive functional groups, and to dispersively mix nanomaterials.

In one embodiment, the method comprises annealing the composite under vacuum and elevated temperature to promote condensation reactions to produce high molecular weight polymer and crosslinking of the nanomaterial to the polymeric matrix. In one embodiment, the cryomilled polymer can be one or more degradable biomaterials, as a multicomponent blend. In one embodiment, the method comprises generating open pores through selective removal of a co-continuous porogen component phase either during manufacture or after implantation.

The increase in strength is attributed to reactive polymer chain ends generated from cryomilling or SSSP, that are maintained during melt molding via bonds with oxidized groups on the nanomaterial surface. For example, annealing the composites under high vacuum at temperatures at or below the melting temperature of the thermoplastic matrix promotes the formation of covalent bonds. The nanomaterials increase the stiffness of the matrix and cause the matrix to resist thermal degradation during extended time above melt temperatures necessary to coarsen interpenetrating polymer networks (IPNs). Their increased matrix stiffness can offset the inherent weakness added by the incorporation of pores, necessary for bone tissue in-growth in a fixation device. Further, in certain embodiments, the nanomaterial acts as nucleation sites for polymer crystallization during manufacture and/or ossification once implanted.

In certain embodiments, the addition of reactive nanomaterial to the mechanochemically processed (e.g., cryomilled) polymer blends can create compatibilizers in-situ during melt molding, molecules that are otherwise costly to produce. Condensation reactions between hydroxyl/carboxylic chain ends and the oxidized groups on the surface of NMs can be enhanced by polymerization below melt temperatures and under vacuum (<10 mmHg) or inert gas (nitrogen) flow, thus facilitating cross-links without the use of a chemical cross-linker. This process can be performed on the dispersed particulate materials after cryomilling, and/or on the finished product after melt molding.

In one embodiment, achieving the initial dispersion in the IPN is not directly a function of the components' viscosities. That is, the present method does not require the viscosity to be as precisely matched to achieve the IPN. Wider variations of material choices may thus be used in the presently described method, compared to those that are otherwise possible in melt blending. Further, the additional of nanomaterial can improve the thermal stability of the network, which is required in certain instances to coarsen an IPN containing one or more degradable biomaterials in order to produce porous devices.

In one embodiment, the devices and methods of the present invention make use of biopolymeric material. Exemplary biodegradable polymers and co-polymers useful in the present device and method include, but are not limited to, polyglycolide or polyglycolic acid (PGA), polylactide or polylactic acid (PLA), poly-L-lactic acid (PLLA), poly-D/L-lactic acid with polyglycolic acid (PDLLA-co-PGA), poly (lactic acid-co-glycolic acid) (PLGA), poly(D,L-Lactide-co-Glycolide) (PDLG), PDLLA, polydioxanone (PDS), poly(ϵ-caprolactone) (PCL), polycaprolactone (PCL) with alginate, polyhydroxybutyrate (PHB), polycarbonate (PC), N-vinyl pyrrolidone copolymers, polyorthoester, chitosan, poly(2-hydroxyethyl-methacrylate) (PHEMA), PEG (polyethylene glycol), and hyaluronic acid. Such polymers may be of natural origin or synthetically produced.

In certain embodiments, the bioabsorbable polymers included in the invention may be processed following similar procedures as those used for thermoplastics. They may be melted and extruded, molded by injection or compression or solvent cast. In certain instances, the presence of moisture must be carefully controlled, because their hydrolytic sensitivity leads to a significant decrease in the material's molecular weight. Therefore, in certain instances, the polymers included in the invention have to be kept completely dry before thermally processing, and its contact with moisture during the processing must be avoided.

Once implanted, biodegradation of the biopolymers included in the invention is mainly caused by hydrolysis of the polymer chain backbone and to a lesser extent by enzymatic activity (Vert & Li, 1992, J. Mater. Sci. Mater. Med. 3:432-446; Li & McCarthy, 1999, Biomaterials 20:35-44). Degradation times depend on multiple factors, such as polymer crystallinity, molecular weight, thermal history, porosity, monomer concentration, geometry and the location of the implant.

Exemplary biopolymers included in the invention comprise PDLG, PLA, PDS, PGA, and PLGA, which are amongst the most commonly used synthetic, biodegradable polymers, with an extensive U.S. FDA approval history (Ella et al., 2005, J. Mat. Sci.-Mat. Med. 16(7):655-662; Huh et al., 2005, Drug Del. Tech. 3(5):52-58).

PGA is a highly crystalline hydrophilic polymer, which tends to lose its mechanical strength rapidly (50% loss over a period of 2 weeks). Upon implantation, PGA degrades in about 4 weeks and can be completely absorbed in 4-6 months (Grayson et al., 2005, Biomaterials 26(14):2137-2145; Ouyang et al., 2002, Mat. Sci. & Eng. C: Biomim. Supramol. Syst, 20(1-2):63-69; Zhang et al., 2006, Pol. Degr. Stab. 91(9):1929-1936; Panyam et al., 2003, J. Contr, Rel. 92(1-2):173-187; Oh et al., 2006, J. Mat. Sci.-Mat. Medicine 17(2):131-137; Valimaa & Laaksovirta, 2004, Biomaterials 25(7-8):1225-1232; Habraken et al., 2006, J. Biomat. Sci.-Pol. Ed. 17(9):1057-1074).

PGA is more hydrophilic than PLA, while PLA has a higher modulus than PGA that makes it more suitable for load-bearing applications. For PLGA and PDLG copolymers, the mechanical strength and the degradation rate depend on the ratio of PLA/PGA. As the content of PLA in the PLGA copolymer increases, the copolymer becomes mechanically stronger and degrades more slowly. In the case of PLA, PLGA, PDLG, and PGA, the final products of the polymer degradation are the acidic monomers (lactic acid and glycolic acid, respectively) that are metabolized to ATP, water and CO₂ (Brady et al., 1973, J. Biomed. Mater. Res. 7:155-166). PLGA degradation is also influenced by other factors including the polymer chain length and characteristics of the surrounding medium.

Chitosan, PHEMA, PEG and hyaluronic acid are biopolymers also included in the invention. They are among the most relevant hydrogels used in the generation of biomaterials. In hydrogels the bonding of hydrophilic macromolecules by means of covalent hydrogen and ionic bonds form a three-dimensional network that is able to retain large amounts of water in their structure. These types of polymers are useful in cartilage, ligaments, tendons and intervertebral disc repair applications (Ambrosio et al., 1996, J. Mater. Sci, Mater. Med. 7:525-530). Chitosan is a weak cationic polysaccharide obtained by extensive deacetylation of chitin and composed essentially of β(1→4) linked glucosamine units together with some N-acetylglucosamine units.

Exemplary nanomaterials that may be used in the devices and methods of the present invention include, but are not limited to, carbon nano-diamonds, detonation nano-diamonds, hydroxyapatite, tricalcium-phosphate, silica, bioglasses, graphene oxides, single-walled carbon nanotubes, multi-walled carbon nanotubes and the like. In certain aspects, carbon nano-materials may provide the functional groups necessary to create cross-links between the nanomaterial and surrounding matrix.

In certain embodiments, the device exhibits enhanced mechanical properties. For example, in certain embodiments, the device has a flexural modulus in the range of about 2.0-4.0 GPa. For example, in certain embodiments, the device has an ultimate stress in the range of about 100-120 MPa. For example, in certain embodiments, the device has a elongation at break in the range of about 5-20%. For example, in certain embodiments, the device has a toughness in the range of about 2-20 MPa/(mm/mm).

In certain embodiments, the devices formed by a method using a combination of mechanochemical processing and vacuum annealing exhibit enhanced mechanical properties as compared to devices formed by a method using only one of mechanochemical processing and vacuum annealing. For example, in certain embodiments, the devices formed by a method using a combination of mechanochemical processing and vacuum annealing have a mechanical property that is 1% greater, 2% greater, 5% greater, 10% greater, 20% greater, 30% greater, 40% greater, 50% greater, 75% greater, 100% greater, 200% greater, 500% greater, or more than the same mechanical property of a device formed by a method using only one of mechanochemical processing and vacuum annealing.

In one embodiment, the nanomaterial comprises nanodiamonds. Nanodiamonds (NDs) are comprised of particles that are about 5 nm in diameter. In one embodiment, the NDs used in the invention vary in diameter from 0.1 nm to 50 nm. In another embodiment, the NDs used in the invention vary in diameter from 0.5 nm to 25 nm. In yet another embodiment, the NDs used in the invention vary in diameter from 1 nm to 10 nm. In yet another embodiment, the NDs used in the invention vary in diameter from 2 nm to 8 nm. In another embodiment, the NDs used in the invention vary in diameter from 4 nm to 6 nm.

In one embodiment, the use of NDs as a nanomaterial within the invention is advantageous because of the high matrix/nanomaterial interface area when the size of the ND particles approaches nanometer domain. By dispersing a mere 1% vol of a nanoparticle of radius ˜2 nm in a polymer (interfacial thickness ˜6 nm), the volume fraction occupied by the interface region is ˜63%, suggesting that more than half of the composite is affected by the presence of the second-phase particles (Winey & Vaia, 2007, MRS Bulletin 32:314-319). Thus, being well dispersed, the NDs included in the invention improve properties of the composites at very low concentrations without compromising the properties of the matrix.

In one embodiment, the ND particles used in the present invention are non-functionalized. It has previously been reported that non-functionalized ND particles tend to form unusually tight aggregates (Krueger, 2008, J. Mater. Chem., 18:1485-1492). Mixing non-functionalized ND particles with a polymer typically results in poor dispersion with micron-sized nanodiamond agglomerates embedded in the matrix. Aggregated ND particles do not produce any property improvement for the composite, acting rather as defects and often leading to deterioration in mechanical properties. However, it is demonstrated herein that the mechanochemical processing of polymer components and ND produces a well-mixed blend.

Even when well-dispersed, however, the NDs act merely as conventional nanofillers with high hardness, performing similar to other ceramic nanoparticles (such as silica or clay) and leading to only moderate improvements in properties. In other words, good dispersion of the nanoparticles in the composite is not sufficient to ensure that the composite will have superior mechanical and thermal properties. A strong interface between the NDs and the matrix must also be present to ensure superior mechanical properties for the corresponding composite.

In one embodiment, the strong interface between the NDs and the matrix is obtained by hydrogen bonds between the matrix and the NDs. In another embodiment, the strong interface between the NDs and the matrix is obtained by covalent bonds between the matrix and the NDs. These bonds are favored because in certain instances, NDs present a large number of functional groups on their surface and are thus able to engage in multiple interactions.

Nanodiamonds included in the invention may present chemical groups on their surface. Such nanodiamonds are generally referred to as “chemically-active nanodiamonds.” Among the methods for generating chemically-active NDs that are contemplated by the invention are air oxidation, hydrogenation, chlorination and ammonia treatment (Mochalin et al., 2009, Mater. Res. Soc. Symp. Proc. 1039, 1039-P11-03).

In one embodiment of the invention, the chemically-reactive NDs are prepared by air oxidation of NDs. Air oxidation (or oxidative purification) affords NDs free of amorphous and graphitic sp²-bonded carbon.

Oxidative purification may be conducted under isothermal conditions using a THM600 Linkam heating stage (Linkam Scientific Instruments Ltd., Tadworth, Surrey, UK) and a tube furnace, and under non-isothermal conditions using a thermobalance (Perkin-Elmer TGA 7, Shelton, Conn., USA). Isothermal experiments include two steps: (i) rapid heating at 50° C./min to the selected temperature and (ii) isothermal oxidation for 5 hours in ambient air at atmospheric pressure. In one embodiment, the temperature range for oxidation of the ND samples investigated is 400-430° C.

Under these conditions, the purity of ND may become comparable to that of microcrystalline diamond. Metal impurities, which are initially protected by carbon shells in the commercial samples, generally become accessible after oxidation and are completely removed by further treatment in diluted acids. In addition to purification, air oxidation dramatically changes the surface chemistry of ND. Oxidation of the nanodiamond particles results in nanoparticles covered by oxygen-containing functional groups such as C═O, COOH, and OH, with a decrease in the content of C—H groups. Carboxyl groups can be easily deprotonated in basic media, thus aqueous suspensions of the oxidized ND have lower aggregation tendencies at pH>7.

In another embodiment of the invention, the chemically-reactive NDs are prepared by high temperature treatment of NDs in H₂ atmosphere. In yet another embodiment, the high temperature treatment of NDs in H₂ atmosphere is for 2 hours at 800° C. This treatment increases the content of C—H-containing groups and completely removes C═O groups as a result of saturation of non-saturated bonds according to reaction (I). H₂ annealing may not significantly remove non-diamond carbon from the sample.

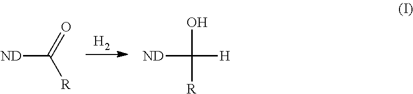

In yet another embodiment of the invention, the chemically-reactive NDs are prepared by chlorine (Cl₂) treatment of NDs for 1 hour at 400° C. This treatment yields acyl chlorides, as shown in reaction (II):

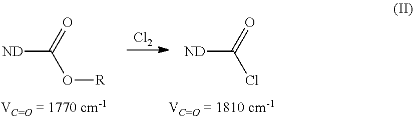

where R is H or a carbon-based group, such as CH₃. Chlorination may also remove carbon from the material due to the formation of volatile CCl₄.

In yet another embodiment of the invention, the chemically-reactive NDs are prepared by ammonia treatment of NDs for 1 hour at 850° C. This treatment may give rise to NH₂-containing groups on nanodiamonds. This treatment may also give rise to C—H, C═N and O—H containing surface functionalities.

In certain embodiments, chemically-active nanodiamonds may be manipulated by standard chemical methods to yield derivatized nanodiamonds, such as surface-functionalized nanodiamonds.

In one embodiment, surface-functionalized nanodiamonds are prepared by chemical modification of chemically-active nanodiamonds. In another embodiment, chemically-active nanodiamonds are themselves surface-functionalized nanodiamonds and are used as such within the invention.

Generation of chemically-active NDs included in the invention may be done in numerous ways, including traditional gas and wet chemistry (Oswald et al., 2006, J. Am. Chem. Soc. 128(35):11635-11642; Mochalin et al., 2007, “High Temperature Functionalization and Surface Modification of Nanodiamond Powders,” In “Materials Research Society Symposium Proceedings,” Boston, Mass., USA, Vol. 1039, No. 1039-P11-03). These methods allow for the generation of chemically-active nanodiamonds with different surface functional groups, which may be used as handles to introduce chemical groups on the surface of the NDs (“surface derivatization”).

In one embodiment, the surface of the chemically-active nanodiamond particles included in the invention comprises carboxylic groups (—COOH). Chemically-active NDs with COOH surface groups have good dispersion stability in aqueous solutions at basic pH (Oswald et al., 2006, J. Am. Chem. Soc. 128(35): 11635-11642). Carboxylic groups on the surface of chemically-active nanodiamonds may be derivatized using methods known to those skilled in the arts.

As a non-limiting example, the carboxylic groups on the surface of chemically-active nanodiamonds may be reacted with an activating agent, such as, but not limited to, EDC (1-ethyl-3-(3-dimethylaminopropyl)carbodiimide), DCC (dicyclohexylcarbodiimide) or DIC (N,N′-diisopropylcarbodiimide), in an inert solvent such as, but not limited to, dichloromethane, tetrahydrofuran or dimethylformamide, and then reacted with a primary or secondary amine, yielding surface-functionalized NDs with immobilized amides.

In one embodiment, the amine is selected from the group consisting of octylamine, decylamine, undecylamine, dodecylamine, tridecylamine, tetradecylamine, pentadecylamine, hexadecylamine, heptadecylamine, dodecadecylamine, nonadecylamine and eicosylamine. In another embodiment, the amine is octadecylamine.

In another non-limiting example, the carboxylic groups on the surface of chemically-active NDs may be reacted with a chlorinating agent, such as, but not limited to, thionyl chloride, phosgene, diphosgene or triphosgene, in an inert solvent such as, but not limited to, dichloromethane, tetrahydrofuran or dimethylformamide, and then reacted with a primary or secondary amine, yielding surface-functionalized NDs with immobilized amides.

In another embodiment, the surface of the chemically-active nanodiamond particles included in the invention comprises amino groups (—NH₂). Amino groups may be introduced on the surface of the chemically-active nanodiamonds by treating nanodiamonds with ammonia at high temperature. Amino groups may also be introduced on the surface of the chemically-active nanodiamonds by attaching bisamines to nanodiamonds containing surface carboxylic groups.

As a non-limiting example, the carboxylic groups on the surface of chemically-active nanodiamonds may be reacted with (i) an activating agent, such as, but not limited to, EDC (1-ethyl-3-(3-dimethylaminopropyl)carbodiimide), DCC (dicyclohexylcarbodiimide), or DIC (N,N′-diisopropylcarbodiimide), in an inert solvent such as, but not limited to, dichloromethane, tetrahydrofuran or dimethylformamide, or (ii) with a chlorinating agent, such as, but not limited to, thionyl chloride, phosgene, diphosgene or triphosgene, in an inert solvent such as, but not limited to, dichloromethane or tetrahydrofuran. The material may then be reacted with a bisamine, in an inert solvent such as, but not limited to, dichloromethane, tetrahydrofuran or dimethylformamide. In one aspect, the bisamine may have both amine groups in unprotected form, in which case the reaction yields an immobilized amide with a free amino group. In another aspect, the bisamine may have one unprotected amino group and one protected amino group, wherein the protective group may be, for example, t-butoxycarbonyl (Boc) or fluorenylmethoxycarbonyl (Fmoc). In this case the reaction yields an immobilized amide with a protected amino group. The protective group may be removed using conditions well known in the art, such as treatment with trifluoroacetic acid or hydrochloric acid in the case of the Boc protective group, or treatment with piperidine in dimethylformamide in the case of the Fmoc protective group. This procedure yields surface-functionalized NDs with amides containing free amines.

An important aspect of be considered in the preparation of nanodiamond-polymer composites included in the invention is the purity level of the starting ND particles. The content of non-diamond phase in as-produced or commercially available NDs may be as high as 75% wt. Purification of as-received or crude NDs using modification methods such as, but not limited to, air oxidation, hydrogenation, chlorination and ammonia treatment, and optional mechanical methods such as, but not limited to, treatment with acidic solutions, results in non-diamond carbon removal and generation of a material with the surface uniformly terminated by specific functional groups. In a non-limiting example, selective air oxidation of as-received ND in controlled conditions may increase the content of diamond phase from ^{˜}25 up to ^{˜1} 95% wt, and convert diverse surface functional groups of non-purified ND into C═O and COOH (Osswald et al., 2006, J. Am. Chem. Soc. 128(35):11635-11642).

In one embodiment, the nanocomposite material comprises 0.001% to 10% of NDs. In another embodiment, the nanocomposite material comprises 0.05% to 5% of NDs. In yet another embodiment, the nanocomposite material comprises 0.1% to 1% of NDs.

A strong interface between the NDs included in the invention and the matrix must be present to ensure improved mechanical properties for the composite contemplated in the invention. One such strong interface may be obtained by forming strong covalent or non-covalent bonds between the NDs and the matrix. In this case, for each polymer matrix, the NDs would contain surface groups capable of forming strong hydrogen bonds or covalent bonds with the molecules of polymer matrix. Covalent bond formation between the purified ND particles and polymer matrix will eventually lead to a material that should fully realize the superior mechanical and thermal properties of ND nanodiamond. In one embodiment, the mechanical processing (e.g., SSSP or cryomililng) of polymer and ND components produces reactive polymer chain ends that can form covalent bonds with oxidized groups on the surface of ND.

In certain embodiments, the present invention provides methods of manufacturing improved fixation devices. In certain embodiments, the method comprises mechanical processing of a biocompatible polymer or polymer blend. For example, in certain embodiments, the method comprises SSSP or cyromilling of the biocompatible polymer or polymer blend. In one embodiment, the method comprises mechanical processing of the biocompatible polymer or polymer blend with a nanomaterial, such as nanodiamonds, HA, bioglass, and the like. In certain embodiments, the method comprises mixing the polymer or polymer blend with nanomaterial to form a composite. In certain embodiments, the method comprises forming a composite comprising about 0.001% to 10% of nanomaterial. In another embodiment, the method comprises forming a composite comprising 0.05% to 5% of nanomaterial. In yet another embodiment, the method comprises forming a composite comprising 0.1% to 1% of nanomaterial.

Mechanical processing is used to disperse the nanomaterial within the polymer or polymer blend, and also to create functional groups on the polymer and/or nanomaterial. Such functional groups may participate in effective covalent bonding of the nanomaterial to the polymeric matrix, thus strengthening the resultant biomaterial. As described herein, mechanical processing of the sample is able to produce biomaterials with enhanced mechanical properties.

The biopolymer, alone or with nanomaterial, may be subjected to SSSP or cyromilling using any known instrumentation known in the art. For example, samples comprising the biopolymer, alone or with nanomaterial, can be cryomilled in cooled grinders or mills, such as those provided by SPEX SamplePrep. In certain instances, cryomilling of the samples is conducted at temperatures less than about −80° C. For example, in certain instances the cyromilling instrumentation is cooled by liquid nitrogen to keep the samples at cold temperature. In certain embodiments, the samples are pre-cooled prior to grinding. In certain embodiments, the samples are processed using SSSP, where the sample is mechanically processed using a twin-screw extruder with cooling zones, which maintains the sample in the solid state during processing. The forces and shear applied to the sample during SSSP is able to create blends and dispersions that are otherwise not possible. Thus, in certain embodiments, SSSP is used to effectively disperse the nanomaterial within the biocompatible polymer or polymer blend.

In certain embodiments, the method comprises annealing the sample. For example, in one embodiment, the method comprises vacuum annealing the sample under low pressure and elevated temperature.

For example, in one embodiment, the samples are vacuum annealed at a pressure of about 0.001 to 20 torr. In one embodiment, the samples are vacuum annealed at a pressure of about 0.05 to 10 torr. In one embodiment, the samples are vacuum annealed at a pressure of about 0.1 to 1 torr. In one embodiment, the samples are vacuum annealed at pressure of about 0.2 torr.

In certain embodiments, the samples are annealed at a temperature at or below the melting temperature of the polymer or polymer blend. The temperature used during annealing will thus depend on the particular polymer(s) of the blend, the relative amount of the polymers within the blend, and the like. In one embodiment, the samples are annealed at a temperature of about 50° C. to about 500° C. In one embodiment, the samples are annealed at a temperature of about 75° C. to about 400° C. In one embodiment, the samples are annealed at a temperature of about 100° C. to about 200° C.

Vacuum annealing of the mechanically processed sample promotes polycondensation reactions between the polymer matrix and the nanomaterial. For example, the polycondensation reactions promote the formation of covalent bonds between the dispersed nanomaterial and polymeric matrix, thereby strengthening the biomaterial.

In certain embodiments, the method comprises molding the samples. For example, the samples may be molded using injection molding, compression molding, or solvent casting. The samples may be molded to produce a biomaterial, for example a fixation device, of any desired shape or size.

In a particular embodiment, the method comprises compression molding of the samples. For example, the samples may be molded at elevated temperature and pressure. For example, in one embodiment, the method comprises compression molding the sample at a pressure of about 2,000 psi. In one embodiment, the method comprises compression molding the sample at a temperature of about 150° C.

In certain instances, the mechanical processing and vacuum annealing of the polymer-nanomaterial composite allows for the composite to withstand thermal degradation that may otherwise occur during molding.

In certain embodiments, the method comprises removal of a sacrificial porogen from the composite, thereby forming a porous biomaterial. In certain instances porous biomaterials, such as porous fixation devices, are preferred as they allow for the improved integration of native tissue into and within the biomaterial. In certain embodiments, porous biomaterials allow for the incorporation of cells, biomolecules, therapeutic agents, growth factors, and the like, into the biomaterial pores.

For example, in one embodiment, the method comprises forming a composite comprising the biocompabible polymer or polymer blend, nanomaterial, and porogen, using the mechanical processing and vacuum annealing procedures detailed above. Porogen removal may be conducted before or after molding of the composite.

In one embodiment, the porogen is a polymeric porogen, including, but not limited to polystyrene, and other thermoplastics soluble in organic solvents such as polyethylene, polypropylene, and polymetheylpentene. Other porogens include, but are not limited to water soluble porogens, such as poly-ethylene glycol, poly-viniyl-alcohol, and various sugars. In certain embodiments, the selection of porogen and the relative amount of porogen in the composite dictates the porosity and/or pore size of the resultant porous biomaterial.

In one embodiment, the method comprises removing the porogen by administering an organic solvent to the composite, which thereby removes the porogen from the composite. Exemplary organic solvents that may be used to remove the porogen include, but not limited to, unsubstituted hydrocarbon solvents with appropriate boiling points, such as cylcohexane, limonene, or water for aqueous soluble porogens.

In certain embodiments, the increased mechanical properties of the biomaterial, due to the mechanical processing and vacuum annealing of the polymer-nanomaterial composite, compensates for the inevitable loss of material strength caused by the formation of pores in the biomaterial. Thus, the present invention allows for the production of porous biomaterials that exhibit mechanical properties strong enough to allow for their use as fixation devices used in various orthopedic procedures, where mechanical strength of the devices are critical for success.

The biomaterials described herein can be used in various medical or surgical applications. For example, in certain embodiments, the present invention provides a fixation device used in various orthopedic procedures. Exemplary fixation devices, include but are not limited to, screws, anchors, plates, pins, rods, staples, and the like. Such devices may be used in procedures such as, bone fracture repair, ligament reconstruction, ligament repair, tendon reconstruction, tendon repair, joint replacement, bone fusion, and the like.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1: Poly(D,L-Lactide-co-Glycolide) composites with functionalized Nano-Diamonds

Described herein are experimental results demonstrating the enhancement of the strength of a degradable surgical fixation devices. Importantly, the data demonstrates that the material can be strengthened to a point where pores can be added for tissue infiltration. Increasing the stiffness and strength of the materials becomes an essential step towards allowing tissue integration to mitigate graft loosening and tunnel widening. The enhanced fixation devices are a result of a novel combined adaptation of diverse processing methods, which enhance the functionality of degradable thermoplastics in fixation devices to include tissue scaffolding. This approach includes combining solid state shear pulverization (SSSP) and solid state polycondensation (SSPC) to both disperse and covalently crosslink polyester thermoplastic biomaterials and detonation surface functionalized detonation nanodiamonds (sfDND). The sfDNDs are enriched with hydroxyl (OH), carboxylic acid (COOH), or amine (NH2) functionalization. Results demonstrate that sfDND-OH embrittle PDLG before annealing and both toughen and strengthen the matrix after annealing with a negative correlation to concentration. PDLG is significantly strengthened at low concentrations (<=0.1% by weight) of sfDND-OH after vacuum annealing.

The fixation strength of a device depends on both its internal and external bonding strengths. The methods described herein are conducted to enhance both the initial fixation strength of the material and its interaction with the cells it will contact. Improving cell adhesion and reducing inflammation could mitigate the effects of graft loosening by tunnel widening. While there have been attempts to integrate nanodiamonds into polylactides, the results were not able to produce covalent bonds with the matrix material. Carbon nanomaterials have been shown to increase the mechanical properties of a matrix if compatibilized (Li et al., 2014, Chemical Engineering Journal, 237: 291-299). The experiments presented herein were conducted to achieve both covalent bonds between polymer crystals and enhance osteoblast attachment. The methods were designed to disperse and covalently link nano-diamonds (ND) to reinforce implant thermoplastics in a manufacturable manner. Solid state shear pulverization is used to disperse the NDs and solid-state poly-condensation is induced under heat and vacuum to bond the NDs to the surface of the cryomilled polylactide/glycolide granules. In certain instances, the material should also be annealed after molding, under vacuum, to ensure continued bonding and crystallization. In order to increase intermediate and long term fixation strength by tissue integration, porous scaffolds are prepared through a phase inversion process wherein polylactide/glycolide, nanodiamond, and polystyrene are cryo-milled to create a uniform distribution before thermally annealing above melt temperatures to grow an open porous structure. Organic solvents are used to remove the sacrificial polystyrene porogen. To decrease the loss of fixation strength of the device often caused by bone resorption around the implant, and to decrease the use of toxic organic solvents, limonene will be used to remove the porogen. Micromolar amounts of this solvent have been shown to decrease the inflammatory pathways associated with osteoclastogenesis and bone resorption.

Materials

Poly-D,L-lactide-co-glycolide (PDLG-8531) was attained from Purac Inc, with an inherent viscosity of 2.93 at acquisition. Raw material was stored under vacuum at −20° C. until use. Functionalized nanodiamonds were purchased from Adamas Inc., 1 gram each in hydroxyl, carboxylic acid, and amine enriched surfaces (ND-OH, ND-COOH, & ND-NH2). Liquid nitrogen was provided by Airgas, Inc.

Cryomilling.

Samples were ground in a SPEX SamplePrep cooled by liquid nitrogen. 6 grams of polymer were loaded into grinding cavity, with or without 6 milligrams of nanodiamond. Samples were pre-cooled for 12 minutes before 15 cycles of 50 seconds grinding at 15 cycles per second and 1 minute of rest time. Milling chamber was rinsed and dried between individual grinds. When triplicates were run, mill was only emptied and refilled between replicates of the same group to evaluate.

Vacuum Annealing.

Samples were dried in a vacuum oven (VWR-1410) connected to a Fisher Scientific Maxima C vacuum pump (model D4B). Temperature measurements were made by a Fluke 51 digital thermometer with a k-type thermocouple. Temperature measurements were made by removing the side access panel of the oven and inserting the thermocouple along the outside of the heated vacuum cavity under fiberglass insulation. Due to hot spots on the floor of the oven, the sample tray was placed atop a wire rack in the center of the oven. Pressure measurements were made by a thermocouple Vacuum gauge (Savant Instruments Inc., VG-5) with a DV-24 vacuum gauge tube (Teledyne). To dry, sample particles were poured into a silicone mold and dried under vacuum overnight at room temperature (26.8° C.). To promote condensation reactions, samples were either heated to a point below their melting temperature (setting 5, wall temperature=90-100° C.), or above melting (setting 7, wall temperature=160-180° C.). Front glass was covered with Styrofoam to insulate, but even at higher temperature setting, the front row of samples barely melted under high temperature.

Compression Molding.

Disks were prepared in a LECO PR-10 Mounting Press equipped with a 1.25 cylindrical mold cavity. The 600 watt heater was controlled with an omega CN7600 PID controller interfaced via RS-485 to a Linux laptop running Python2.7 to script parameters and log temperature data. Samples were compressed during heating at 50° C. to 2,000 psi, no subsequent pressure adjustments were made through the duration of testing.

Sectioning.

Samples disks were cut to perform a variety of characterization processes on a Buehler Isomet-1000 diamond saw with a 6 inch diameter blade that is 0.5 mm thick (No. 11-4276). Samples were cooled while cutting with DI water. Sample disks/cylinders were sectioned vertically into 2 mm thick increments to create beams for mechanical testing.

Mechanical Testing.

A Bose Electro-force was used to perform 3 point bend with a 100-lbf load cell. Beams 2 mm thick by 6 mm tall, were placed over a span of 2 cm. Displacement rate was constant at 1 mm/minute, where data logging began at contact force of 0.02 lbs and were each loaded until failure. Force and displacement data was collected at a constant rate of 10 Hz and manually stopped when the specimen broke.

Flexural Strain was calculated as:

$${σ}_{f} = \frac{3 \cdot F \cdot L}{2 \cdot W \cdot {H}^{2}} \qquad ( 1 )$$

Flexural Strain was calculated as:

$${∈}_{f} = \frac{6 \cdot D \cdot h}{{L}^{2}} \qquad ( 2 )$$

Data analysis was performed in Matlab R2014b. The failure point was determined as the minima of the second order derivative of the stress-strain curve. Ultimate stress was determined as the maximum stress observed in each curve. Flexural modulus was determined by smoothing the data with a moving average lowpass filter (5 elements wide), and taking the minimal points of the first derivative.

FTIR Spectroscopy.

Principle Component Regression (PCR) and Partial Least Squares Regression (PCR) were used to correlate Fourier Transformed Infrared Spectroscopy in Attenuated Total Reflectance (FTIR-ATR). Matlab R2015a was used to analyze the FTIR-ATR data. All sets were converted from absorption to transmission, normalized per group, and smoothed with Savitsky-Golay filtering.

Cell Culture & Microscopy.

CellSegm (Matlab toolbox) was used to process the confocal image stacks to find cell number and size on scaffold.

The results of experiments are now described.

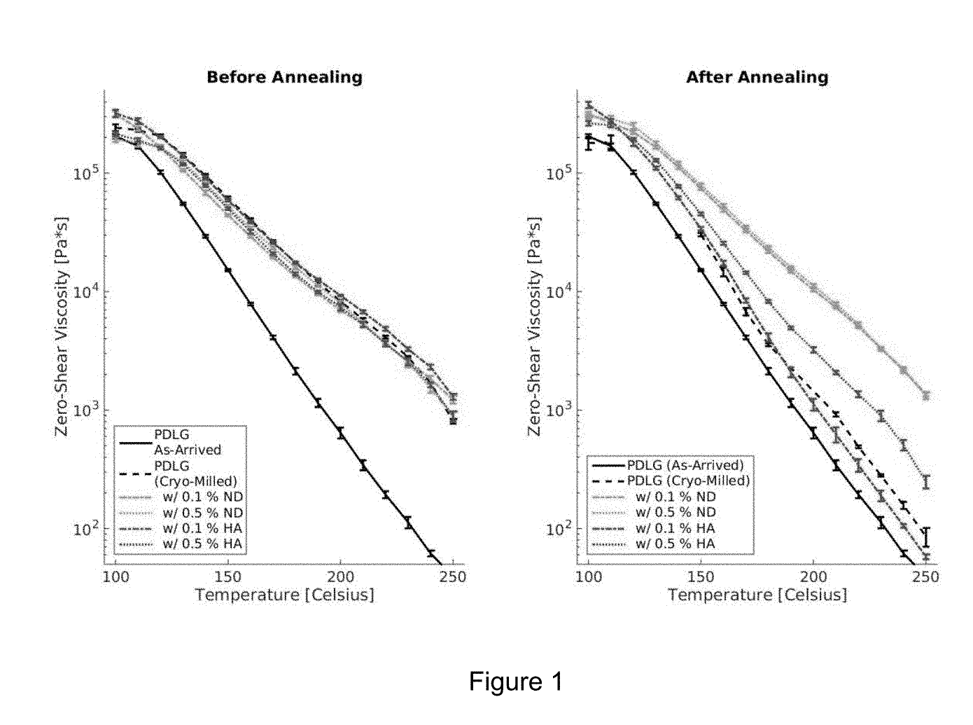

Initial experiments using rheometry to compare PDLG versus PDLG cryomilled alone or with nanocomposites (nanodiamond or hydroxyapatite) demonstrates that cryomilling increases the zero-shear viscosity of the material, and that annealing affects the zero-shear viscosity of the material (FIG. 1).

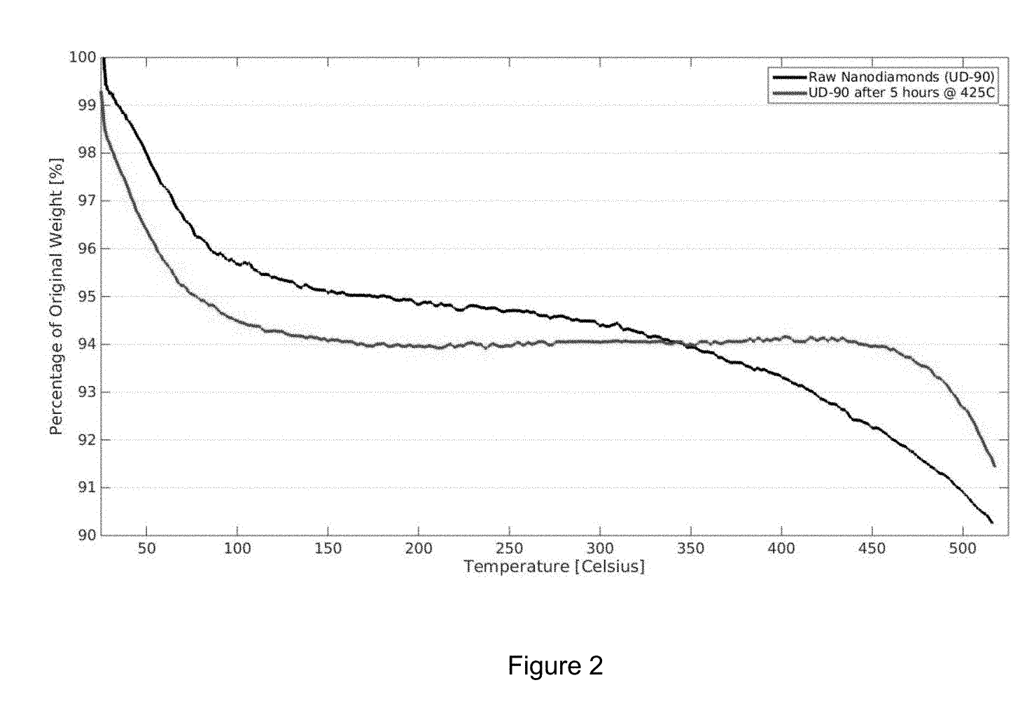

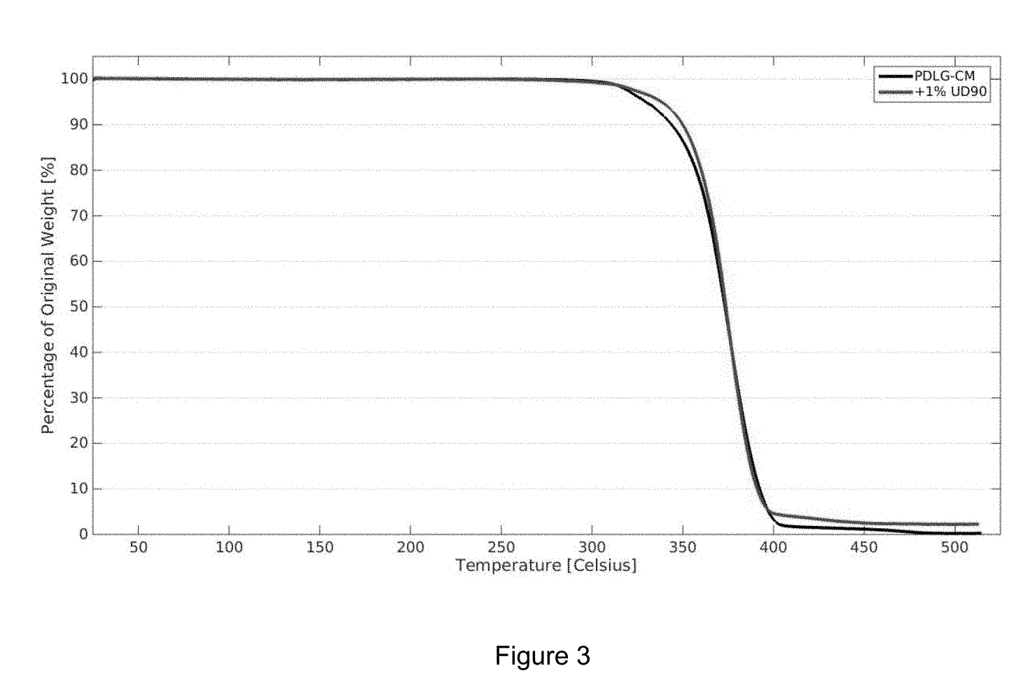

Thermogravimetric analysis (TGA) of raw nanodiamonds versus sintered nanodiamonds is shown in FIG. 2, while TGA of cryomilled PDLG versus cryomilled PDLG+1% nanodiamond is shown in FIG. 3.

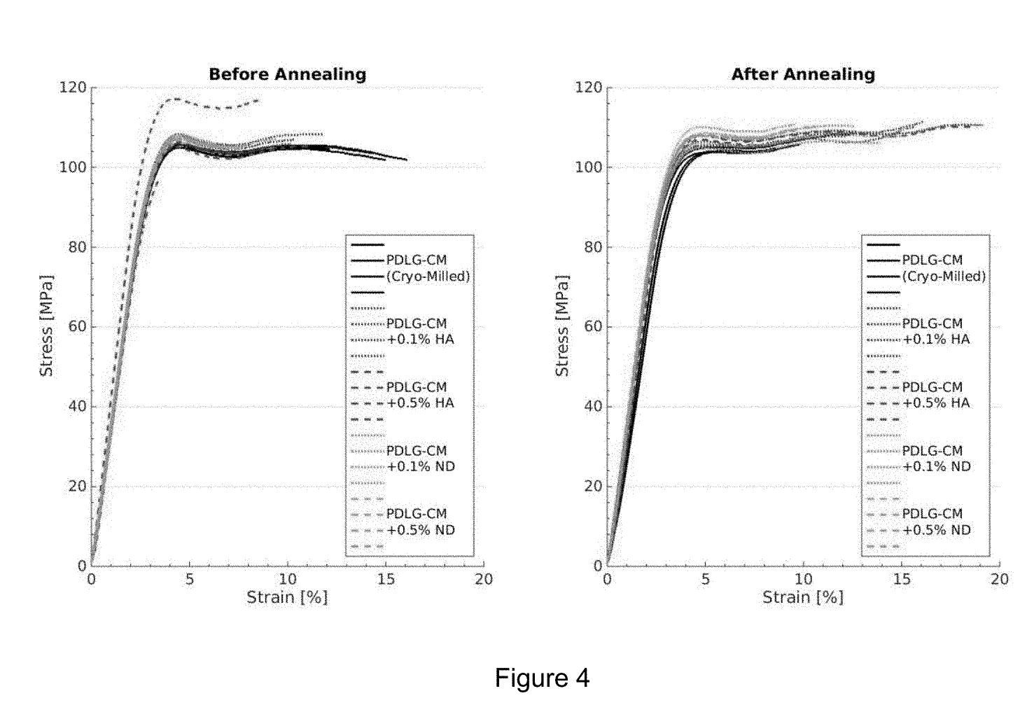

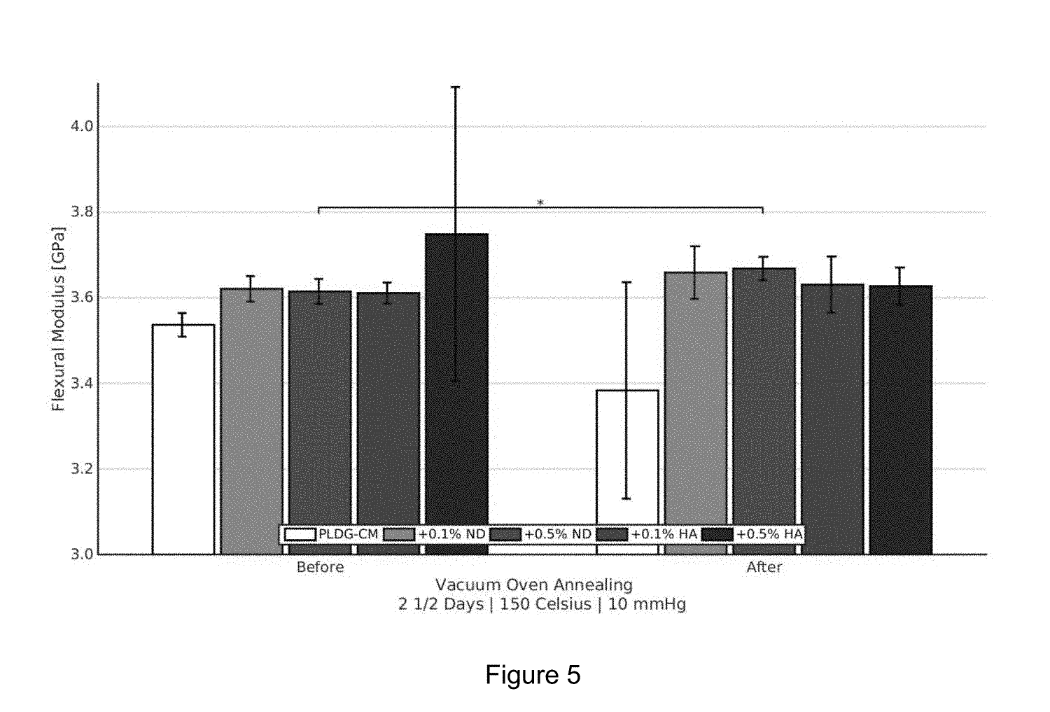

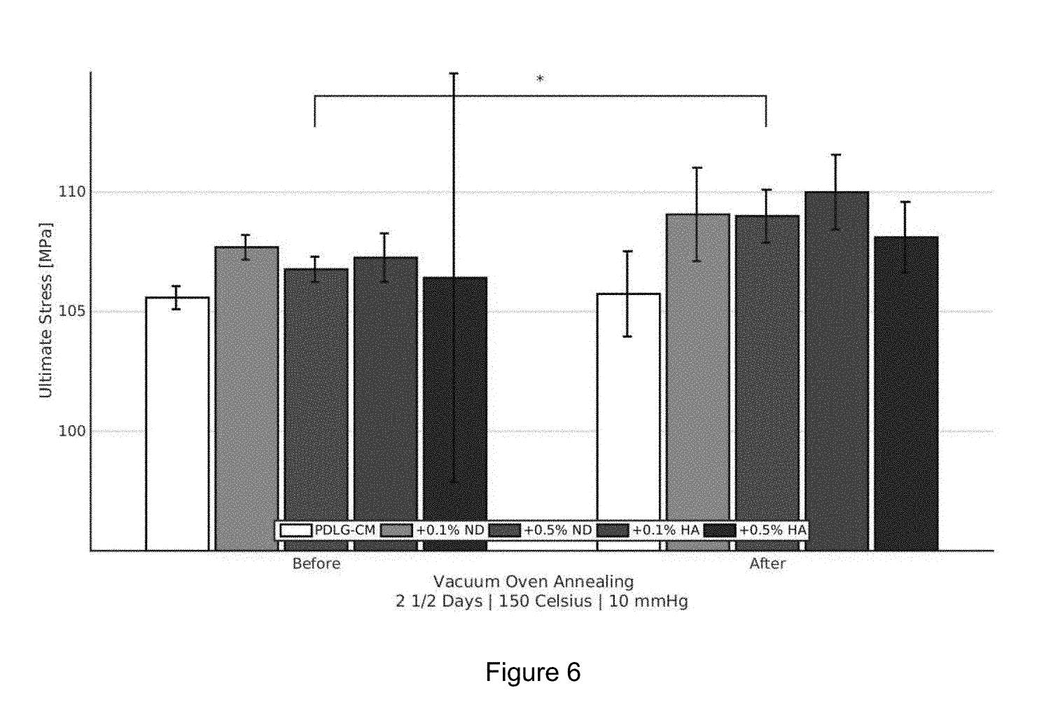

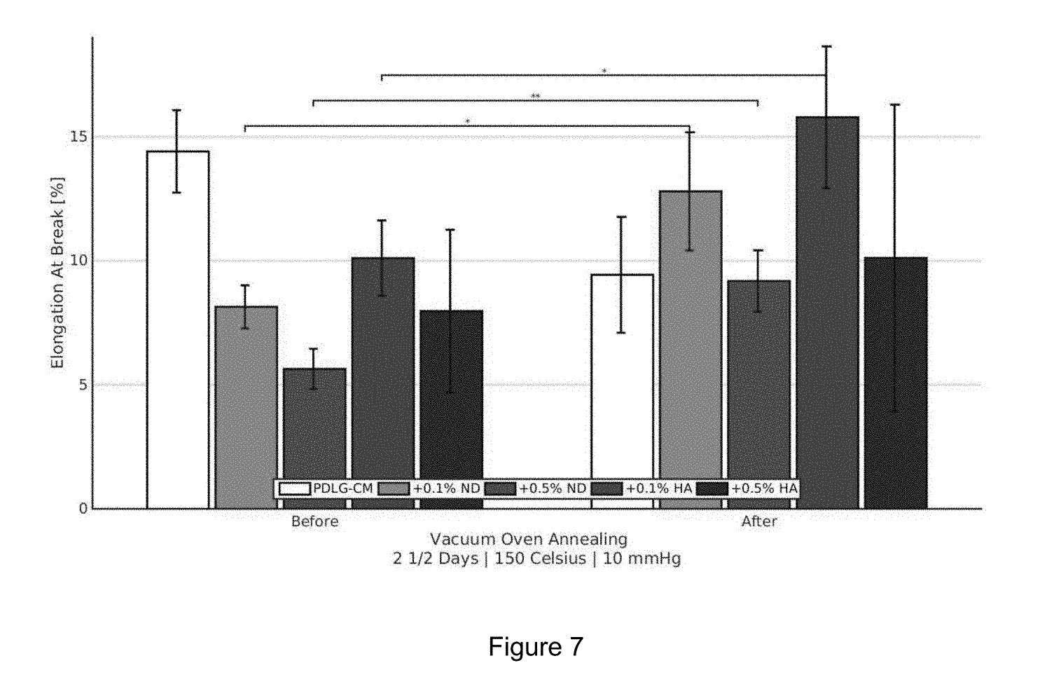

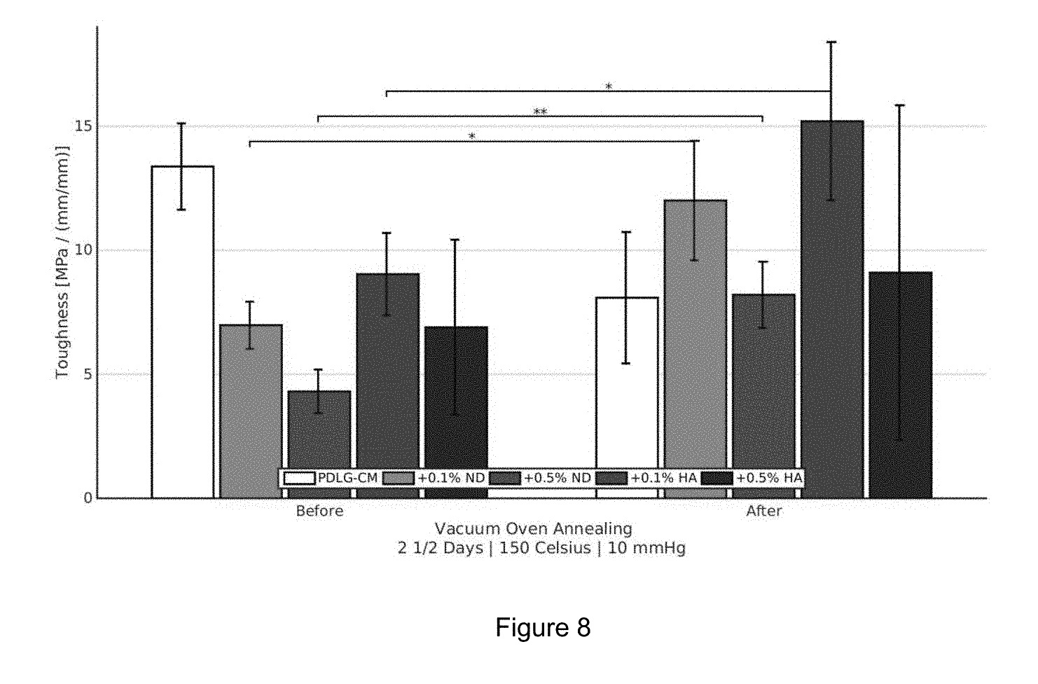

Experiments using flexural testing to compare PDLG cryomilled alone or cryomilled with nanocomposites (nanodiamond or hydroxyapatite) (FIG. 4) demonstrates that cryomilling with nanocomposites increases stiffness (FIG. 5), ultimate stress (FIG. 6), elongation at break (FIG. 7) and Toughness (FIG. 8). Further, it is demonstrated that annealing also increases mechanical parameters of the material (FIG. 4-FIG. 8).

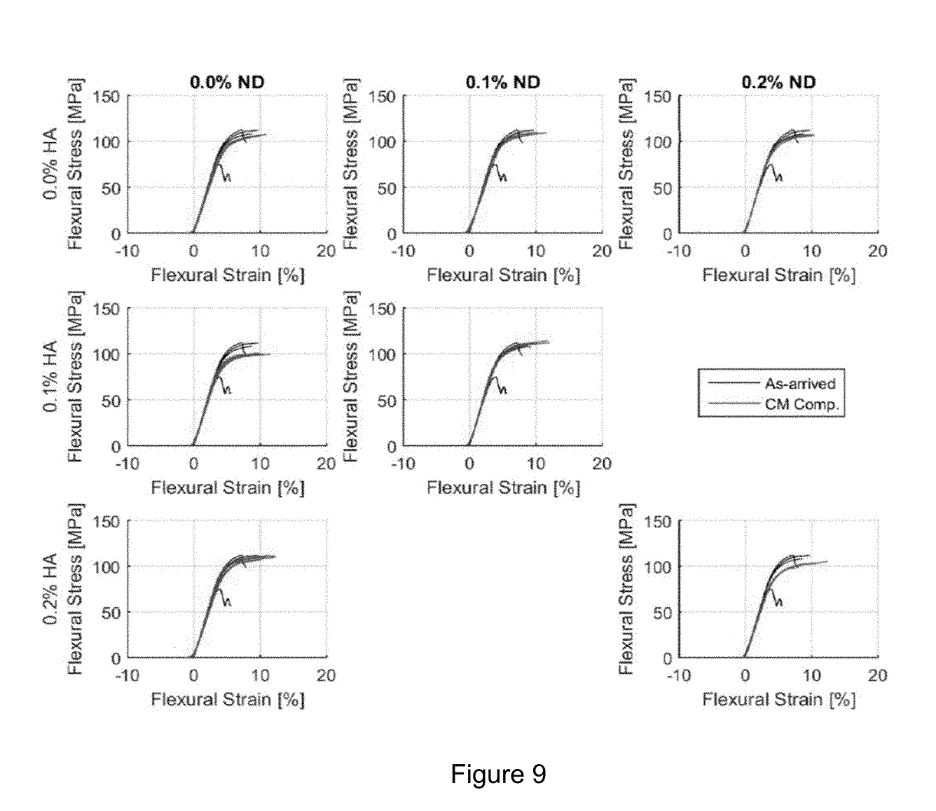

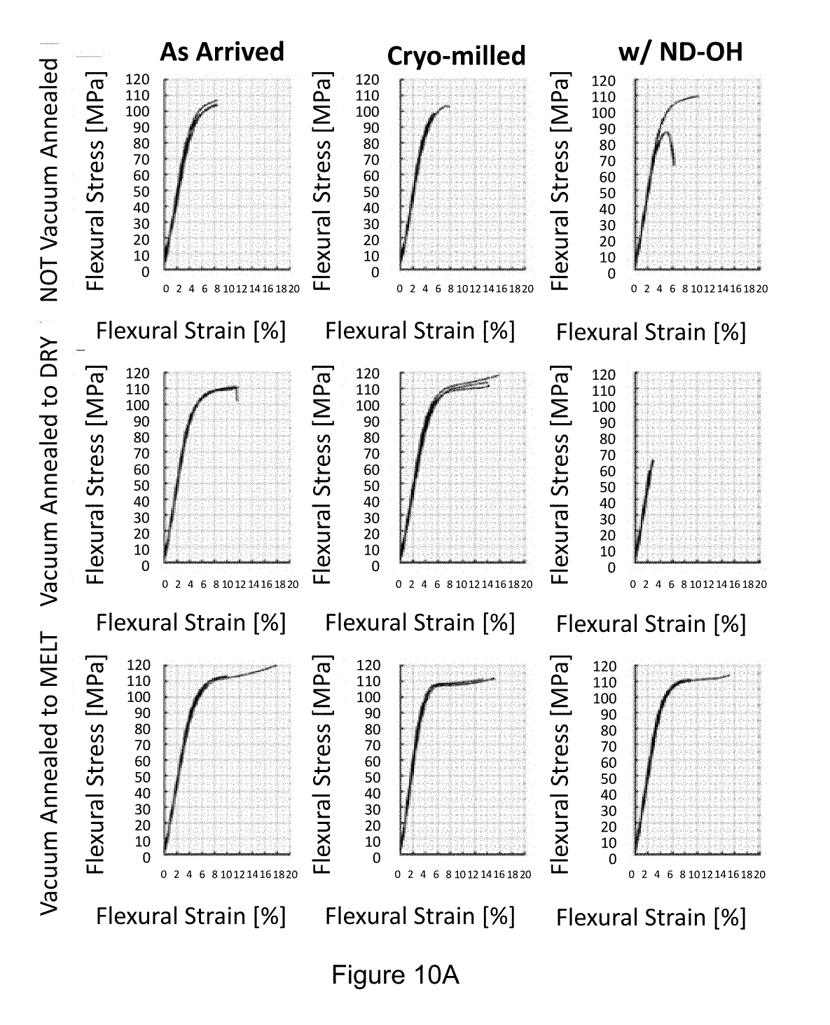

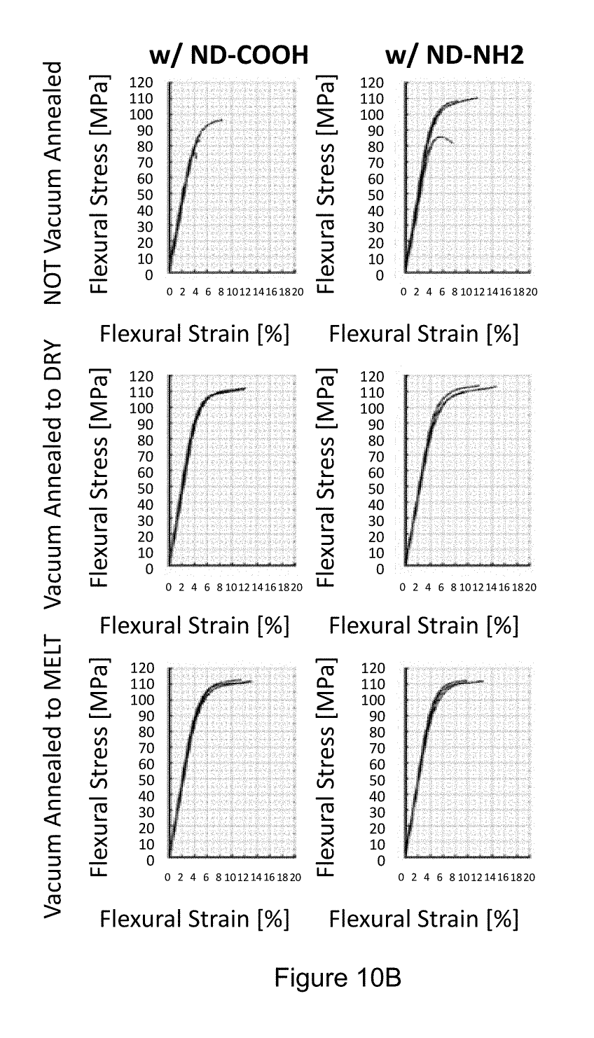

Flexural testing was also performed on cryomilled PDLG8531 with ND and/or hydroxyapatite (HA), which demonstrated that all cryomilled material exhibited superior mechanical properties as compared to non-cryomilled PDLG8531 (FIG. 9). Further testing was done to compare various vacuum annealing procedures (not vacuum annealed, anneal to dry, and annealed to melt) on PDLG8531 only dried under vacuum at room temperature, PDLG8531 vacuum dried and then vacuum annealed above melt temperature, or PDLG8531 cryomilled before being vacuum dried and then vacuum annealed above melt temperature.

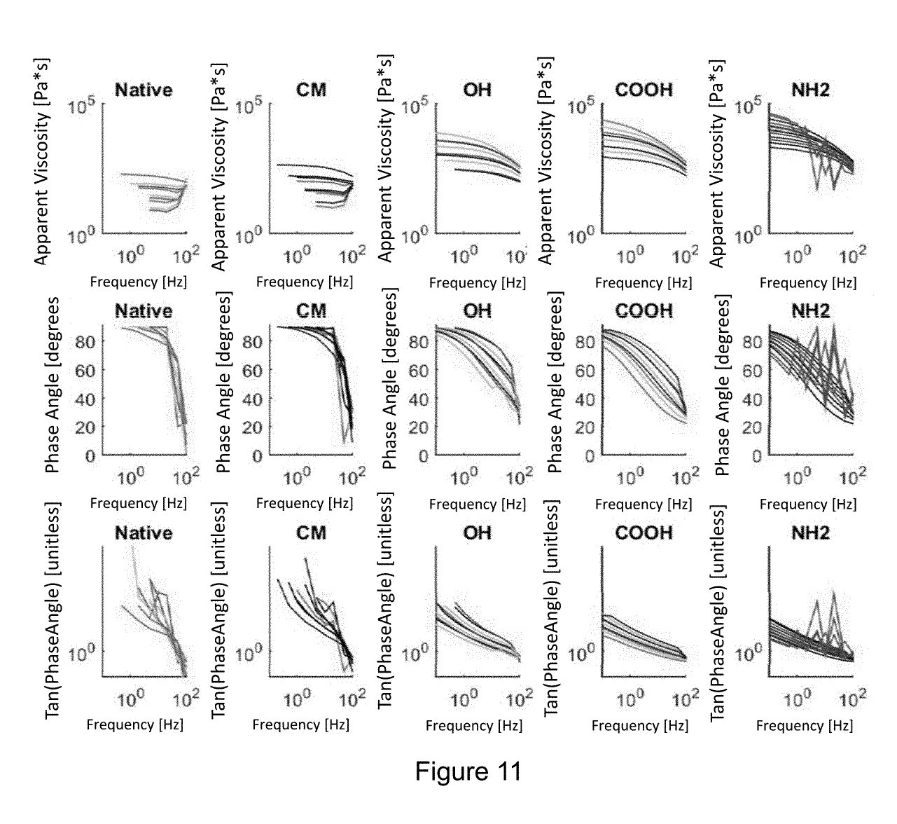

Rheometry of 5 types of samples, all vacuum annealed for 72 hours at 150 Celsius & 0.2 Torr is shown in FIG. 11. Native samples were just annealed, CM (Cryomilled) samples were milled in the SPEX sample prep, and the OH/COOH/NH2 samples were cryomilled with 0.1% of functionalized nanodiamond. The first row represents apparent viscosity as a function of oscillatory frequency. Subsequent rows are derived from this first row.

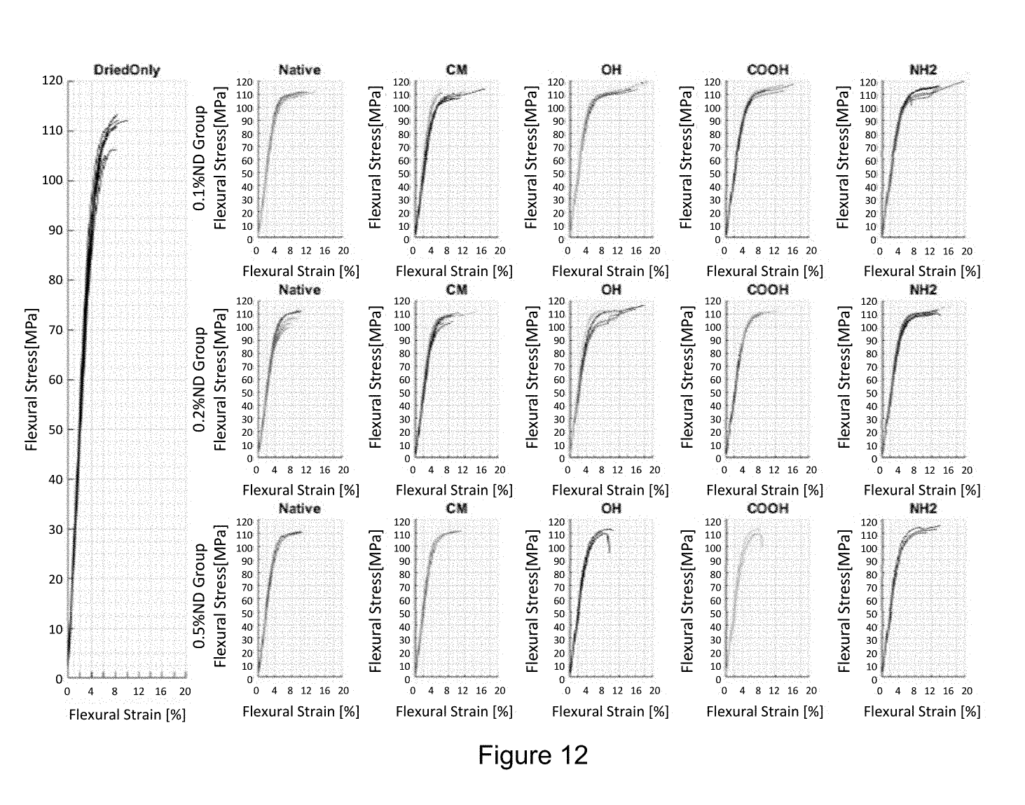

Three point bend tests were done on beams of native or cyromilled PDLG material to produce stress-strain graphs, from load to failure (FIG. 12).

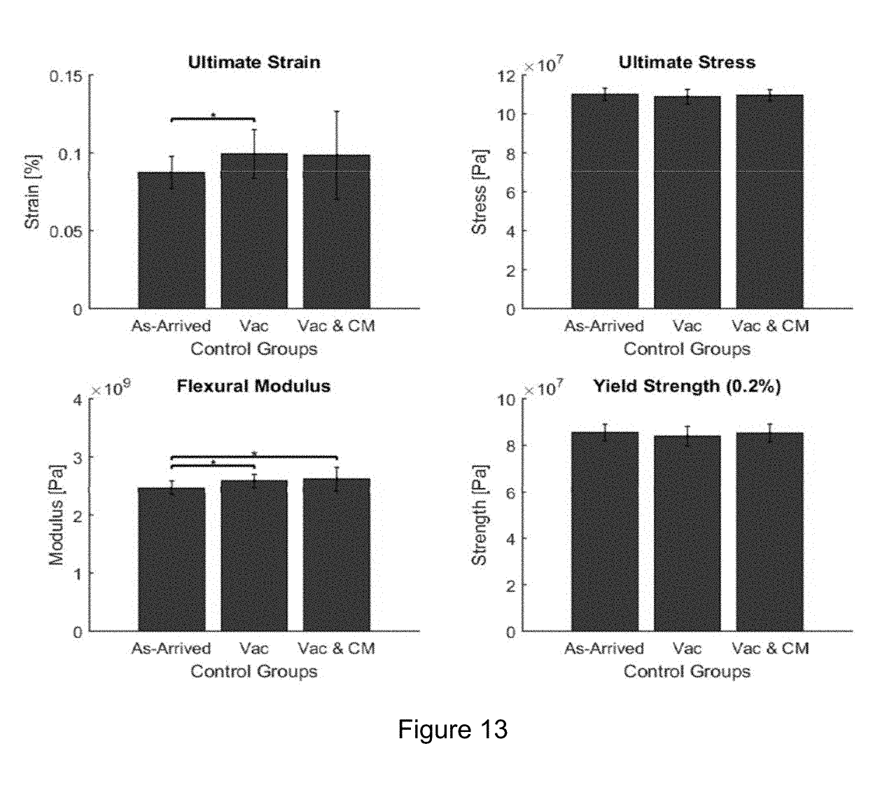

The comparison of processing procedure steps as the affect the mechanics of the final material product is shown in FIG. 13. When comparing material alone (dried under high vacuum at room temperature), vacuum annealing at 150 Celsius & 0.2 Torr for 72 hours, and Cryomilled and Vacuum annealed, it is observed that vacuum melt annealing alone both toughens and stiffens the material.

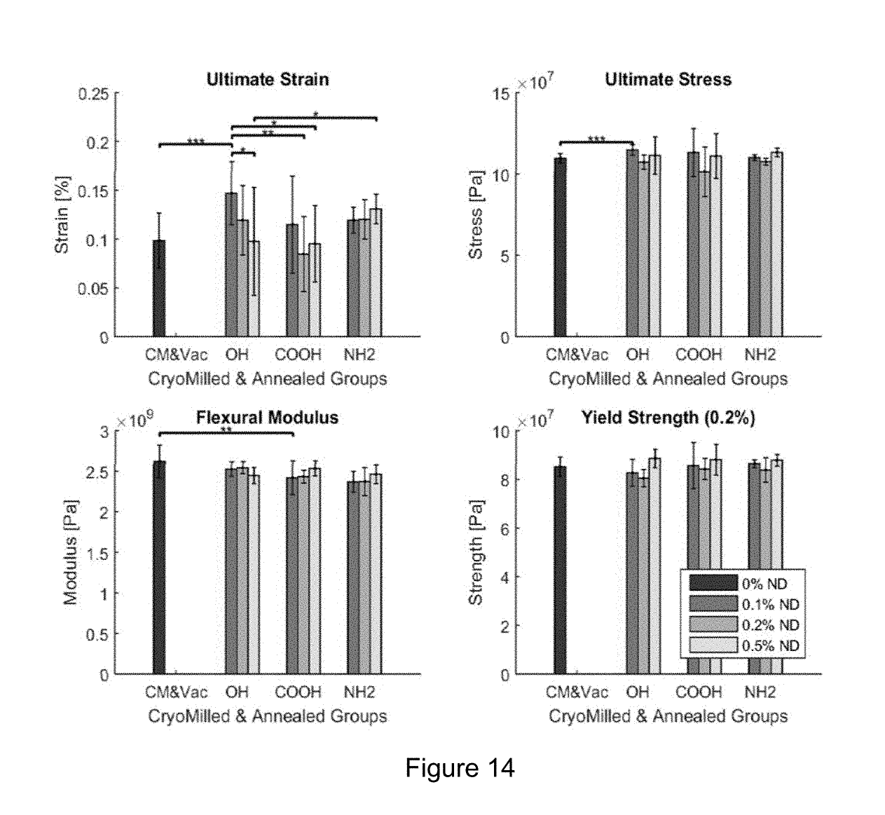

The comparison of mechanical parameters of PDLG alone versus cryomilled and annealed with various concentrations of functionalized ND is shown in FIG. 14.

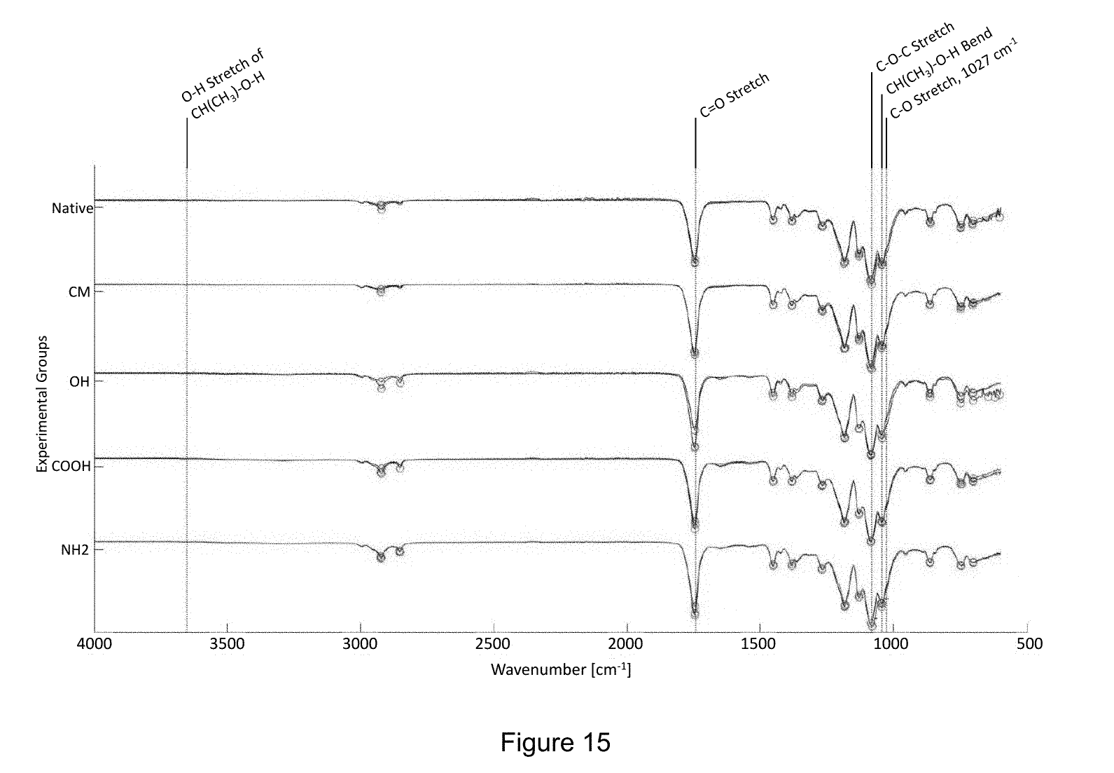

FTIR-ATR Transmission peaks after normalization and Savitsky-Golay smoothing for the native PDLG versus cyromilled PDLG alone or with functionalized ND is shown in FIG. 15.

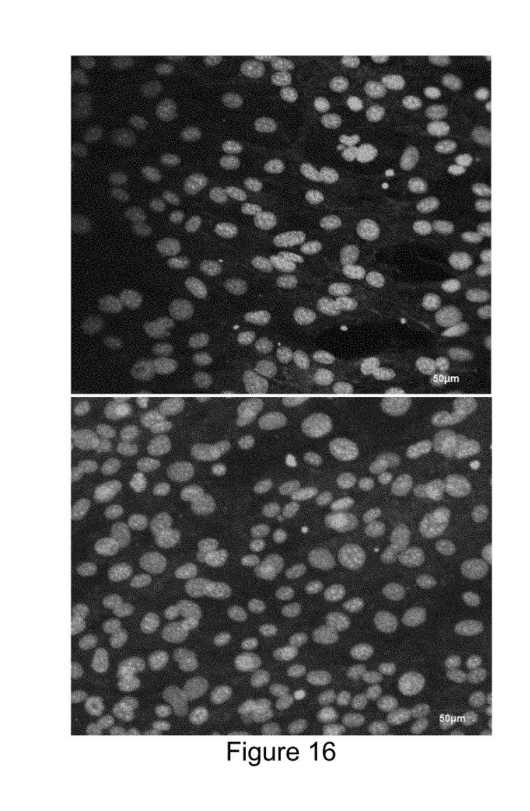

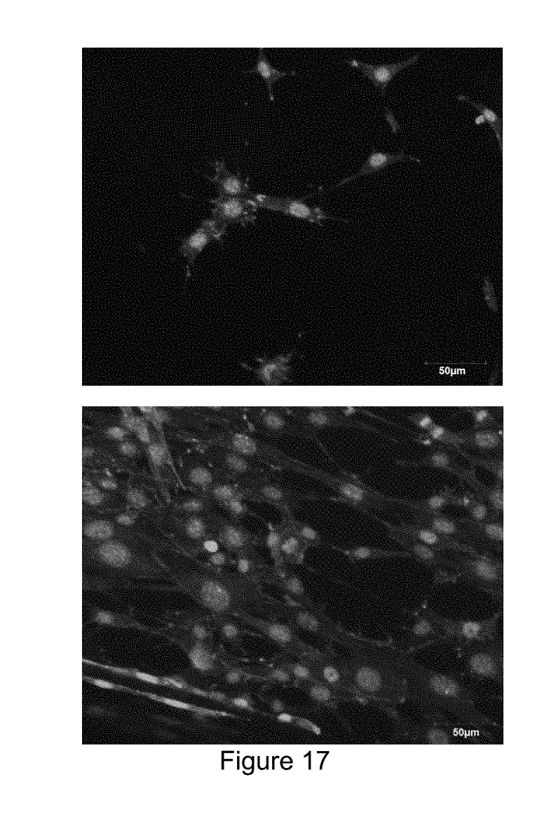

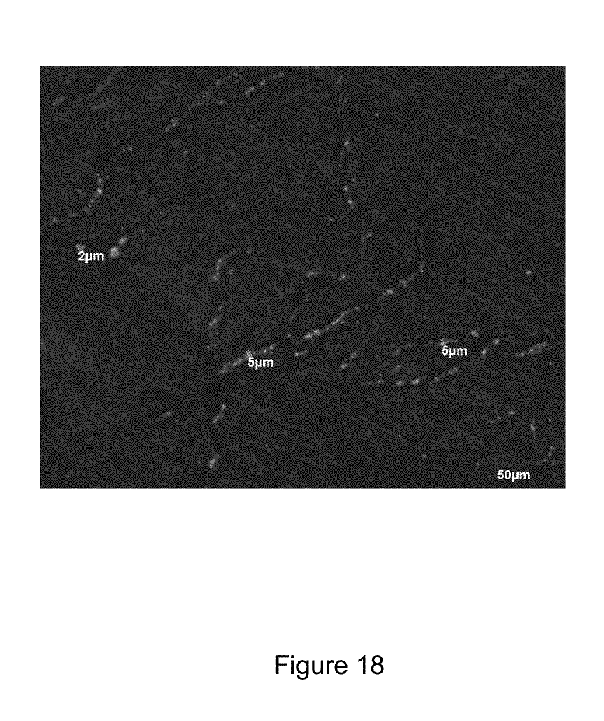

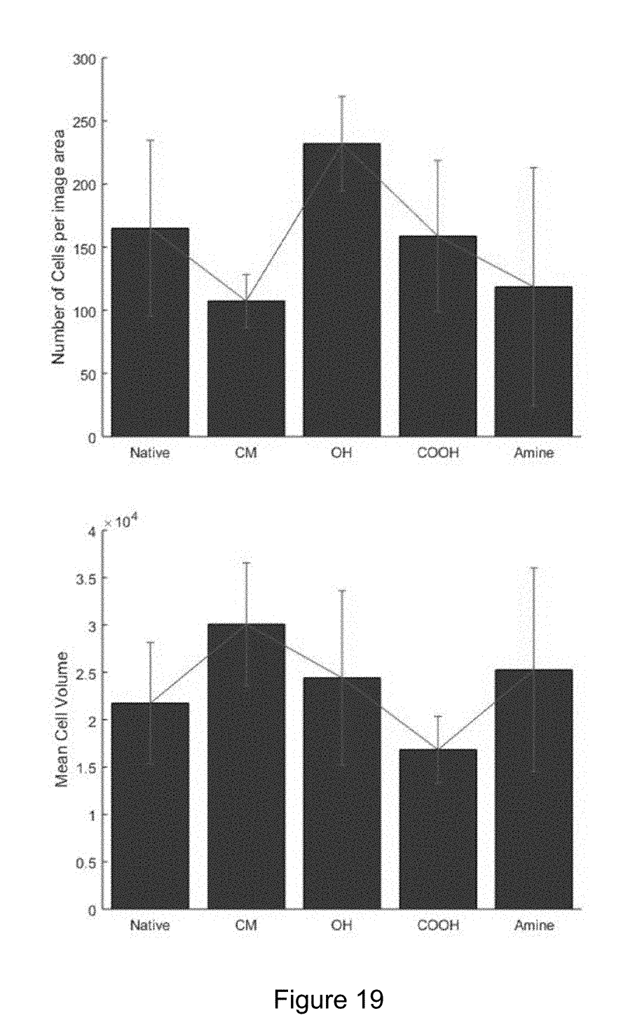

7F2 osteoblasts were cultured on native PDLG, cryomilled PDLG8531 (FIG. 16), and cyromilled PDLG with amine functionalized ND (FIG. 17). Amine functionalized nanodiamonds could even be seen with the cytoskeletal fluorescent dye (FIG. 18) along the boundaries of the original polymer granules. Upon quantification, it is observed that material comprising cyromilled PDLG and hydroxyl functionalized ND exhibits enhanced cell support (FIG. 19).

The results indicate that the hydroxyl groups not only inhibit thermal degradation shown in amine and acid functionalized NDs, but they actually strengthen the polymer. Without polycondensation, the polymer was embrittled by the ND-OH, and after polycondensation the results indicate that low amounts of only the hydroxyl group significantly increases the strength of the composite. There also appears to be a dose dependent decrease in general mechanics (both stiffness and ultimate strength) with increasing ND content. Only the Hydroxyl functionalized ND showed improved modulus and ultimate strength.

### Example 2: Nanodiamond Composites

Described herein are experimental results demonstrating the maximization of the mechanical reinforcement potential of degradable polyesters traditionally used in monolithic implants by providing ND only in strategic locations and ensuring their surface moieties can interact with the matrix polymer (such as by having the polymer grafted to the nanoparticle).

Cryogenic milling, a form of solid state shear pulverization (SSSP), has already been demonstrated to dispersively and distributively mix micronized particulates for interpenetrating polymer network production (J. B. Jonnalagadda et al., 2014, J. Mech. Behav. Biomed. Mater. 40C:33-41; R. M. Allaf et al., 2011, J. Mater. Sci. Mater. Med. 22:1843-53; J. B. Jonnalagadda et al., 2015, J. Biomater. Appl. 30(4):472-83; R. M. Allaf et al., 2015, J. Appl. Polym. Sci. 42471); the aim of the following study is to leverage this production step to additionally coat the polymer granules with a very thin layer of ND to reinforce grain boundaries (J. Masuda et al., 2008, Macromolecules. 41:5974-5977; U.S. Pat. No. 8,734,696). Additionally, annealing polylactide (PL) above their T_{g} and below their Tₘ, under high vacuum of dry nitrogen flow, can cause polycondensation to create new covalent bonds as water is drawn away. The following study attempts SSPC under heat and high vacuum to bond the NDs to the surface of the cryomilled (CM) polylactide/polyglycolide (PL/PG) granules (W. Li et al., 2014, Chem. Eng. J. 237:291-299). Porous scaffolds are prepared through a phase inversion process wherein polylactide/glycolide, nanodiamond, and polystyrene are cryo-milled to create a uniform distribution before thermally annealing above melt temperatures to grow an open porous structure. Organic solvents (cyclohexane) are used to remove the sacrificial polystyrene porogen.

Carbon nano-materials (CNMs) generally fall into three categories: nano-tubes, graphene oxides, or nano-diamonds (ND). Of these groups, NDs have the highest cellular uptake and the least cytotoxicity (X. Zhang et al., 2012, Toxicol. Res. (Camb). 1:62). CNMs may increase biocompatibility with current synthetic tissue sca□olds (J. S. Czarnecki et al., 2015, Clin. Podiatr. Med. Surg. 32:73-91). Polylactide has already been covalently bonded with oxidized CNMs, such as graphene oxide (L. Hua et al., 2010, Polym. Degrad. Stab. 95:2619-2627). CNM composites can bind more surface proteins to decrease platelet adhesion and subsequently immunogenic responses (A. M. Pinto et al., 2013, Colloids Surfaces B Biointerfaces. 104:229-238). MSC expression of Integrin αᵥ was a□ected by the presence of graphitic carbon on titanium implants, independent of surface roughness (R. Olivares-Navarrete et al., 2015, Biomaterials. 51:69-79). MSCs seeded on carboxylated multiwalled carbon nanotubes increase their viability and ALP activity over both PLGA alone and tissue culture plastic (C. Lin et al., 2011, Colloids Surfaces B Biointerfaces. 83:367-375). Carbon may not be the only nanomaterial capable of increases the sti□ness and strength of polylactide. Small amounts of nano-hydroxyapatite particles may act as nucleation sites for crystallization and e□ectively increase the sti□ness of a composite biomaterial (C. Delabarde et al., 2010, Compos. Sci. Technol. 70:1813-1819; S. I. J. Wilberforce et al., 2011, Polymer (Guildf). 52:2883-2890). When properly exfoliated, carbon nanomaterial composites (such as graphene oxide) should not exceed a weight percent of approximately 1% (H. Fang et al., 2013, Macromolecules. 46:6555-6565). Annealing in the presence of these nanoparticles as nucleators can significantly increase the sti□ness of the material (S. I. J. Wilberforce et al., 2011, Acta Biomater. 7:2176-2184). CNMs tend to act as nucleating agents in PLLA composites (H. Wang et al., 2011, Thermochim. Acta. 526:229-236). Kumar et al provides a useful method for CNMs compounded with polyester biomaterials (S. Kumar et al., 2014, RSC Adv. 4:19086). Beyond nucleation, functionalized CNMs have the potential to both increase bonding between polymer chains of the matrix material and increase the hydrophilicity of the biomaterial surface (O. J. Yoon et al., 2011, Compos. Part A Appl. Sci. Manuf. 42:1978-1984). During degradation, the loss of ductility is primarily associated with decreasing molecular weights (C. R. M. Roesler et al., 2014, Polym. Test. 34:34-41); therefore ductility may be increased by preserving crosslinking between the polymer chains.

CM has been shown to increase the sti□ness of a polymer matrix, by increasing crystallinity through increased nucleation (M. Henry, Solid-state Compatibilization of Immiscible Polymer Blends: Cryogenic Milling and Solid-state Shear Pulverization, Bucknell University, 2010). CM/SSSP has also been shown to generate free radicals that can create branched polymers or compatibilizers in situ (A. H. Lebovitz et al., 2002, Macromolecules. 35:8672-8675; D. Feldman, 2005, J. Macromol. Sci. Part A Pure Appl. Chem. 42:587-605). The formation of covalent bonds between ND and the polymer matrix are possible (M. Modesti et al., Effect of Processing Conditions on the Morphology and Properties of Polymer Nanocomposites, 2009). Oxidized CNMs have already been shown to exhibit some amount of bonding when dispersed in a PLA matrix. Covalently bonding linear chains of the thermoplastic matrix to the surface of CNMs has been shown to significantly toughen such a composite (W. Li et al., 2014, Chem. Eng. J. 237:291-299). Oxidized CNMs have also been shown to increase the cell attachment to PLA and reduced platelet activation (A. M. Pinto et al., 2013, Colloids Surfaces B Biointerfaces. 104:229-238). Surgical fixation devices made from bioresorbable composites, like hydroxyapatite (HA)/poly-L-lactic acid (PLLA), can reduce the severity of fibrous tissue and increase calcification (H. Akagi et al., 2014, J. Biomater. Appl. 28:954-62). Though, HA composites are merely dispersed and not covalently bound. Transesterification in melt and free radical crosslinking have been demonstrated as a compatibilization methods in PLA blends, resulting in increased elongation at break (M. B. Coltelli et al., 2010, Polym. Degrad. Stab. 95:332-341; M. B. Coltelli et al., 2011, Polym. Degrad. Stab. 96:982-990).

Detonation nanodiamonds are produced from detonating high explosives (with a low oxygen balance) in a closed vessel with gaseous N₂ and CO₂, and liquid or solid H₂O (V. N. Mochalin et al., 2012, Nat. Nanotechnol. 7:11-23). The result of this process is a heterogeneous population diamond clusters and graphitic carbon; the graphitic soot can be removed through high heat in the presence of air (S. Osswald et al., 2006, J. Am. Chem. Soc. 128:11635-42). The nanodiamonds themselves are a heterogeneous population of polyfunctional surface features, which can be fractionated by ultracentrifugation (I. Larionova et al., 2006, Diam. Relat. Mater. 15:1804-1808).

In order to understand if there is any relationship between the surface chemistry of the ND and their matrix materials, specially functionalized ND were acquired from Adamas Nanotechnologies (see Error! Reference source not found.). ND with three types of surface functionalities were purchased from Adamas: hydroxyl groups (ND-OH), carboxylic acid (ND-COOH), and amine (ND-NH₂).

TABLE 1

| | Detonation nanodiamond powder: | | | |
|---|---|---|---|---|
| | Hydroxylated | | Carboxylated | Anime |
| Abbreviation: | ND-OH | | ND-COOH | ND-NH₂ |
| Purity: | >98% cubic phase | | | |
| Primary particle size: | 4-5 nm | | | |
| Average aggregate size: | 60-80 | nm | 60 nm | |
| Zeta Potential: | +30 | mV | −45 mV | +20 mV |

| Ash Content: | 0.6% | | |
|---|---|---|---|
| Amount purchased: | 1 g each | | |
| Price: | $75 | $35 | $100 |

A goal of the following study is to analyze the parameters associated with milling and dispersing a ND composite: ND type versus percentage. The primary criteria for success is derived from the load to failure in mechanical testing. Three sets of milled samples were annealed at 0.1, 0.2, and 0.5% ND concentration with each functionalization and controls for the annealing and Cryomilling processes. The presence of any nanoparticles can create nucleation sites within a polymer, subsequent crystallinity changes could independently affect stiffness (C. Delabarde et al., 2010, Compos. Sci. Technol. 70:1813-1819). Mechanics, rheometry, and FTIR are used to look for signs of bonding changes within the types of composites.

The materials and methods are now described.

Materials

All nanodiamond composite experiments used the same polymer source: Poly-D,L-lactide-co-glycolide (PDLG-8531) was attained from Purac Inc., with an inherent viscosity of 2.93 ^{dl}/_{g} at time of acquisition. Raw material was stored under vacuum at −20° C. until use. Surface functionalized ND were purchased from Adamas Nanotechnologies Inc.; 1 g each of ND-OH, ND-COOH, and ND-NH₂ (Error! Reference source not found.). Liquid nitrogen was provided by Airgas, Inc.

Cryomilling

Sample were placed 6 grams at a time into the milling vessel. Unless specifically stated otherwise, nanodiamond composites in this section contain 0.1% of a functionalized ND (i.e. 6 mg ND to 6 grams PDLG-8531). This is the lowest concentration possible with equipment resources at hand, without performing serial dilution of prior millings. Milling parameters were 12 minutes pre-cool, followed by 15 cycles of 50 seconds at 15 CPS with 1 minute intervals.

Vacuum Annealing

Samples were dried in a vacuum oven (VWR-1410) connected to a Fisher Scientific Maxima C vacuum pump (model D4B). Temperature measurements were made by a Fluke 51 digital thermometer with a k-type thermocouple. Temperature measurements were made by removing the side access panel of the oven and inserting the thermocouple along the outside of the heated vacuum cavity under fiberglass insulation. Due to hot spots on the floor of the oven, the sample tray was placed atop a wire rack in the center of the oven. Pressure measurements were made by a thermocouple Vacuum gauge (Savant Instruments Inc., VG-5) with a DV-24 vacuum gauge tube (Teledyne). To dry, sample particles were poured into a silicone mold and dried under vacuum overnight at room temperature (26.8° C.). To promote condensation reactions, samples were either heated to a point below their melting temperature (setting 5, wall temperature=90-100° C.), or above melting (setting 7, wall temperature=150-160° C.). Front glass was covered with Styrofoam to insulate, but even at higher temperature setting, the front row of samples barely melted under high temperature.

Compression Molding

Samples were shaped for mechanical testing using a LECO PR-10 with a 1¼ inch diameter cylindrical mold cavity. The heater was originally controlled by a manual dial, refined temperature control was attained by removing the internal temperature dial from the heater unit and replacing it with a PID controller (Omega CN7600) with relays to control a power strip and a k-type thermocouple. An RS-485 to USB adapter was used to integrate the controller with a laptop running Linux (Ubuntu) and python 2.6. The PID control system was used to heat samples to a peak heat of 200° C. for 15 minutes before returning to room temperature for demolding.

Sectioning

All samples were wafered on a Buehler Isomet-1000 diamond saw with a 6-inch diameter, 0.5 mm thick blade (No. 11-4276), at a cutting speed of 800 rpm and a counterweight of 100 grams. Sample widths varied between 100 μm to 2 mm depending on analytical test.

Mechanical Testing

A Bose Electroforce was used to perform flexural load to failure using a 3-point bend rig with a span of 20 mm and a load cell of 100 lbf. Sample beams were 2 mm thick by 6 mm tall. The axial displacement was set constant at 1 mm/minute, where data logging began at a contact force of 0.02 lbs and loaded until failure. Displacement rate was constant at 1 mm/minute. Force and displacement data was collected at a constant rate of 10 Hz and manually stopped when the specimen broke.

Flexural stress was calculated as:

σf=(3*F*L)/(2_W_H2)

Flexural Strain was calculated as:

εf=(6_D_h)/L2

Data analysis was performed in Matlab R2014b. The failure point was determined as the minima of the second order derivative of the stress-strain curve. Ultimate stress was determined as the maximum stress observed in each curve. Flexural modulus was determined by smoothing the data with a moving average low-pass filter (5 elements wide), and taking the minimal points of the first derivative. Using the same flexural rig, cyclic loading until failure was also performed. Using force feedback control, sinusoidal oscillations of either 40 MPa or 80 MPa were performed until failure.

Rheometry

Samples were sectioned from blocks after the vacuum annealing procedure. Rheological measurements were made on a dynamic rheometer (ATS Rheosystems) with 25 mm parallel plates. Strain-controlled oscillatory stress sweep testing regimes were used on account of the visco-elastic nature of a thermoplastic during melting, wherein strain oscillations were constrained to 1% for frequencies of 10⁻¹ s⁻¹ to 10⁺² s⁻¹. A minimum of 3 oscillations were performed at each frequency step. Relevent processing temperatures were evaluated for both polylactides and polystyrenes; a sweep of seven temperatures were studied for each sample (n=3) in each group, from 150° C. to 250° C. in steps of 20° C. The gap between the parallel plates was zeroed at 200° C., before reducing the stage temperature to 150° C. and loading the sample. Gap height was set to 0.9 mm, samples were trimmed at 10% above. The temperature dependence of zero shear rate viscosities and phase angle measurements were used to determine optimal porogen selection and annealing temperatures.

### Imaging of ND Distribution

Brightfield imaging was utilized to demonstrate the distribution of nanodiamonds within the composite structure. Sections were wafered to 100 μm thick. Polarized light microscopy is also presented for samples that have undergone tensile test until failure. Sample dimensions were 6 mm by 2 mm in rectangular cross section, and 20 mm in length.

FTIR Spectroscopy

Principle component (PC) regression and partial least squares (PLS) regression were used to correlate reactive groups by Fourier-transformed infrared spectroscopy (FTIR) in attenuated total reflectance (ATR) mode (32 scans per reading, 3 readings per sample, 3 samples per group). All Spectral data was collected between 600 and 4000 cm′. Matlab R2015a was used to analyze the FTIR-ATR data. All sets were converted from absorption to transmission, normalized per group, and smoothed with Savitsky-Golay (width of 9 cm⁻¹).

Degradation

Degradation studies were conducted on the best performing ND composite alongside HA and unfilled polymer controls, wherein samples of various sizes were kept in 50 mL of αMEM (+10% FBS+1% PennStrep) for 9 weeks. On retrieval samples were rinsed with 3× washes of DI water and gently agitated, dried in a chemical hood for 4 hours, then dried overnight under 0.2 Torr vacuum. Mass differentials were used to quantify degradation.

Mineralization

Porous samples, with and without 0.1% ND-OH, were kept in simulated body fluid (SBF) for 1 week before being rinsed gently in DI water (3 times) and dried overnight at 0.2 Torr vacuum. Mineralization effects were observed via SEM/EDS and MIR Spectroscopy.

The results are now described.

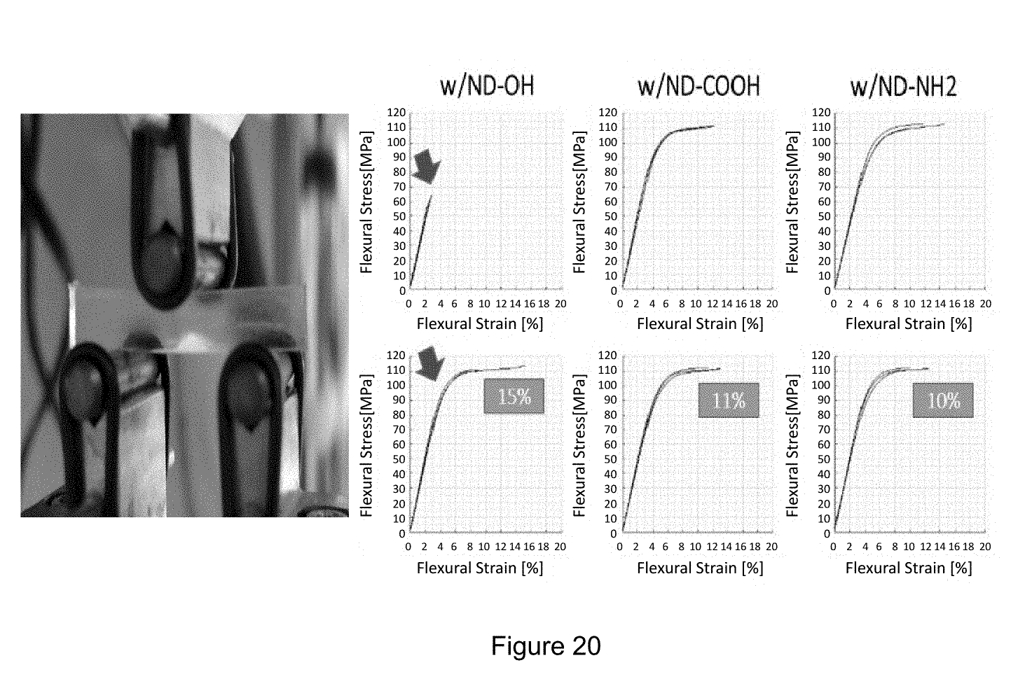

In order to understand whether a polymer composite is worth using, a functional improvement must first be demonstrated. Since the goal is to increase the integrity of a polymer matrix, mechanical testing is the most important result to discuss first. On the left panel of FIG. 20 is the testing setup used for the mechanical analysis. The right hand panel of FIG. 20 shows the stress strain curves for the various ND composites. Of note, the bottom row has an additional vacuum Oven Annealing (OA) step to induce SSPC (48 hours at 150° C. and 0.2 Torr). The nature of the stress-strain curves does not noticeably change before or after SSPC for the ND-COOH or ND-NH₂ composite groups. In contrast, the ND-OH composites are embrittled before SSPC, and significantly increases strain at failure without sacrificing stiffness or yield strength.

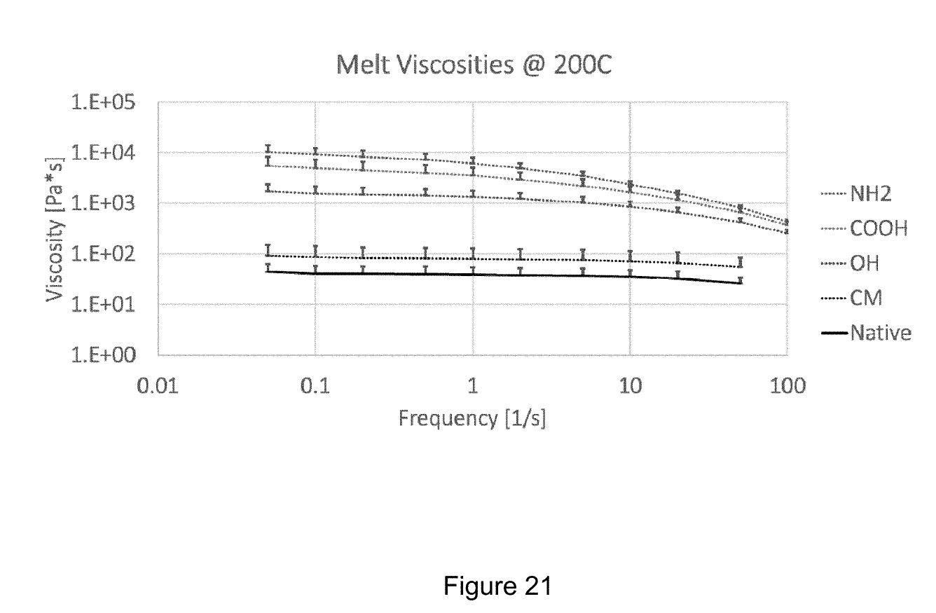

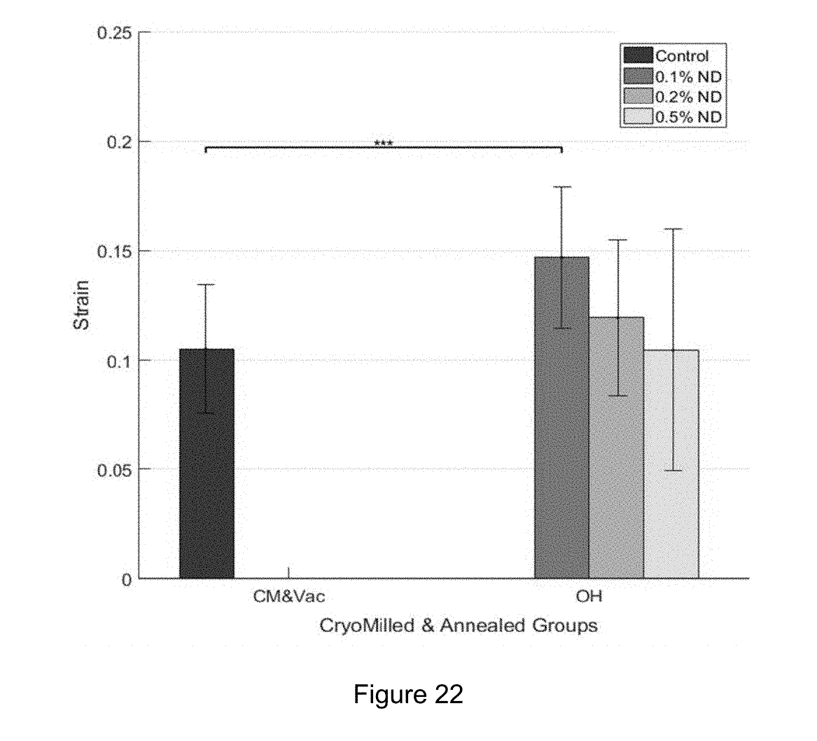

Once an optimal thermoplastic blend was determined, the feasibility of making a product from this material was then investigated. In order to create an implant from a thermoplastic, viscosity measurements are needed to demonstrate how the material flows in order to fill a mold cavity and form the end user's required geometry. Rheological comparisons can help to ascertain how flowable a thermoplastic is and how easy it will be to fill compression or injection mold cavities. Polylactide CM′ d with various functionalized NDs, 0.1% by weight, (OH, COOH, & NH2) and vacuum annealed for SSPC, are rheologically compared to controls of CM without ND (CM) and neither CM or ND (native). SSPC was still performed on controls. As seen in FIG. 21, the CM process itself increases viscosity over the native control at all observed frequencies; this result is cohesive with other literature that claims SSSP can fracture polymers in a way that generates free radical and reactive crosslinks (i.e. chain branching) (US Pat. App. No. 2014/0364577; J. Kim et al., 2008, Polymer (Guildf). 49:2686-2697; A. H. Lebovitz et al., 2002, Macromolecules. 35:9716-9722). All of the ND composites in FIG. 21 show substantially increased viscosity curves over the two controls; the hydroxyl functionalized nanodiamond (ND-OH) has by far the least detrimental increase in viscosity by nearly an order of magnitude compared to the other ND composites. Thus, ND-OH is the most processable of the ND composites evaluated.

The next step is to understand the relationship that content has on these qualities. Rheometry has an obvious positive correlation with ND-OH content (not shown), wherein increasing ND increases viscosity. The strain at failure demonstrated a negative correlation with ND-OH content. The effect of ND-OH content was parametrically analyzed by varying the weight percentage in a log spacing; the experiments shown in FIG. 20 were repeated with 0.1%, 0.2%, and 0.5% ND-OH. Only the lowest amount of ND-OH significantly improved strain at failure, with a negative correlation to increase weight percentage. Lower concentrations are worth investigating, but not feasible with the mass balance and cryomill available for this study.

Another discovery was ND-OH appears to be more resilient to cyclic fatigue even at yield stress. Yield stresses were not affected by the presence of 0.1% ND-OH.

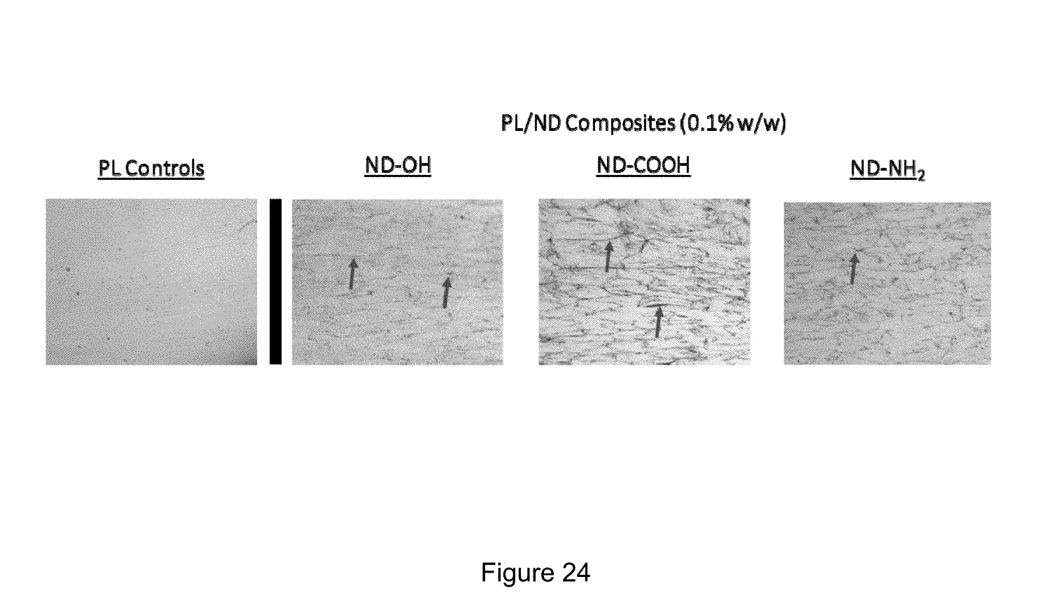

The reason for why ND-OH is superior to the other ND-COOH and ND-NH2 when reinforcing PL can be demonstrated from comparing the zeta potentials found in Error! Reference source not found. and the dark borders visible around the polymer granules of the ND composites visualized in FIG. 24: ND-OH: most positive zeta-potential, least visible borders; ND-COOH: most negative zeta potential, darkest borders; ND-NH2: median zeta potential, intermediately borders.

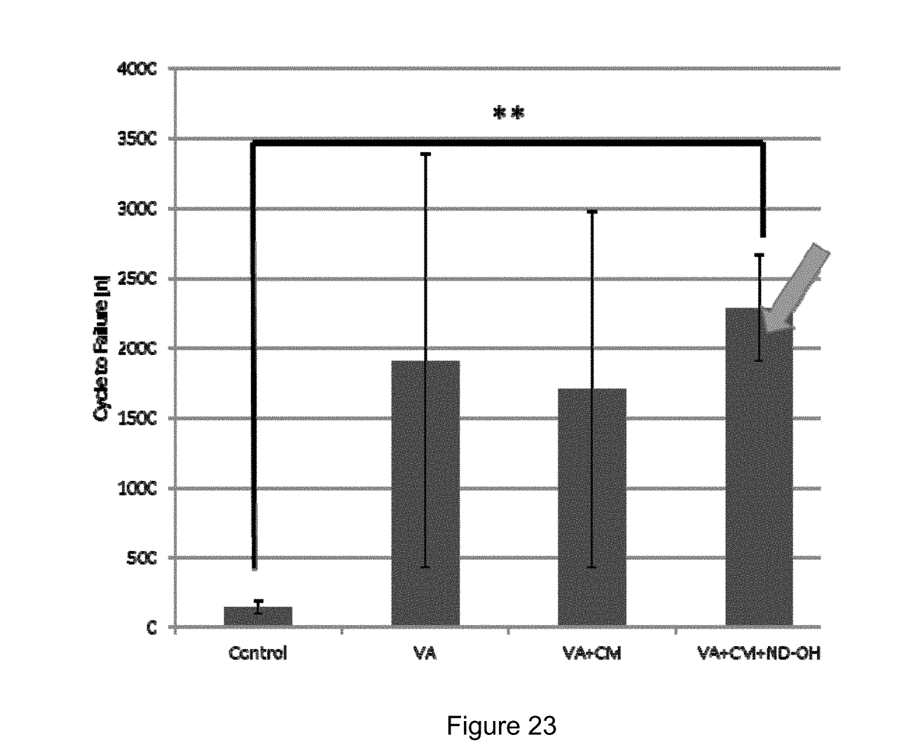

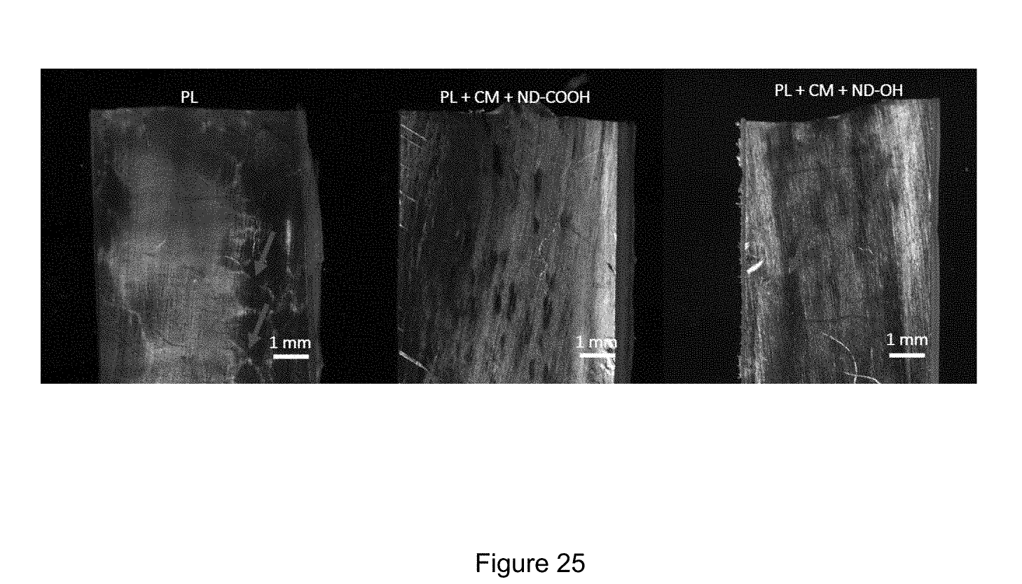

Having now visualized the agglomeration of ND on the borders of polymer domains, the negative correlation between increasing ND content and decreasing strain at failure is clear: clusters of ND can interfere with bonding between polymer domains. In order to test the hypothesis of load sharing between polymer domains, across the ND that cluster at polymer grain boundaries, thin sections of the composites were loaded to failure under uniaxial tension at 1 mm/minute. These thin sections were then visualized under polarized light, after failure, to reveal patterns of strain induced birefringence (FIG. 25). In the left image of virgin PL dried and molded, the large granules barely stick to each other; narrow stress risers lead to rapid failure, which was also observed in this sample group during fatigue testing in FIG. 23. Although the middle image is specifically of PL+(CM+ND-COOH)+SSPC, the result is representative of the groups with either no ND or no ND-NH₂; these groups share generally good stress distributions on account of milling and annealing, but there are still well defined black spots that are representative of large polymer particles not participating in load distribution (these dark spots are not bubbles or voids). Finally, the 0.1% ND-OH group, with CM and SSPC, have once again shown superior load distribution; the boundaries of the dark spots are less defined or blurred, indicating a gradient of birefringence and increased load sharing.

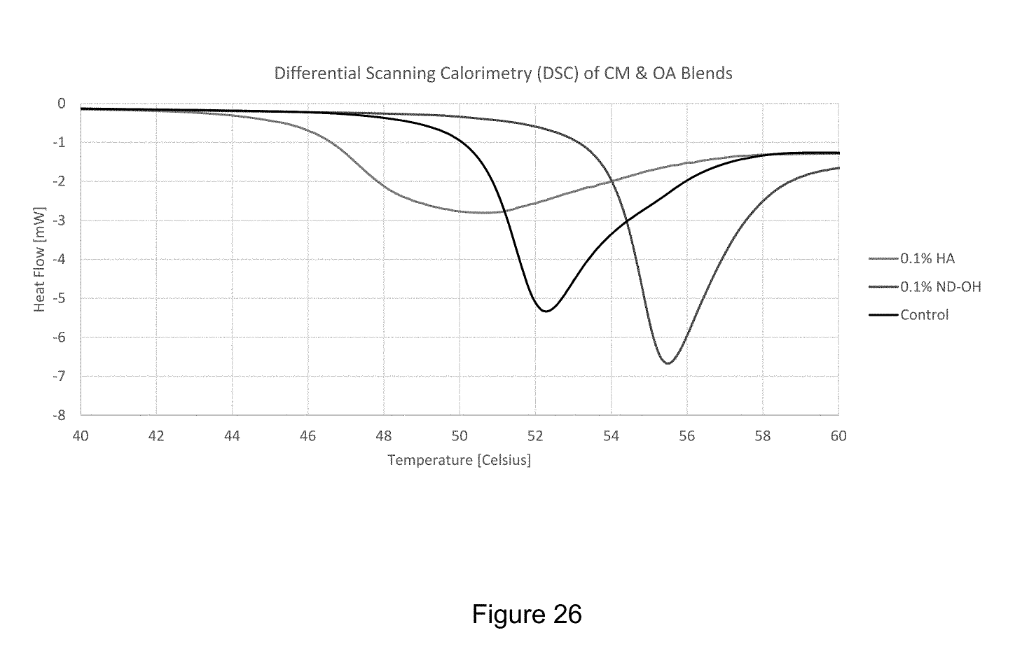

Significant research has also shown that nanomaterials can act as nucleation sites, increasing crystallinity during annealing and in turn increasing the mechanical integrity of the PL matrix (C. Delabarde et al., 2010, Compos. Sci. Technol. 70:1813-1819; H. Wang et al., 2011, Thermochim. Acta. 526:229-236; C. Delabarde et al., 2011, Polym. Degrad. Stab. 96:595-607; S. Saeidlou et al., 2012, Prog. Polym. Sci. 37:1657-1677; J. Z. Liang et al., 2013, Compos. Part B Eng. 45:1646-1650; J. Liuyun et al., 2014, Compos. Sci. Technol. 93:61-67; M. Nerantzaki et al., 2014, Polym. Degrad. Stab. 108:257-268; H. Wang et al., 2012, Thermochim. Acta. 527:40-46). For this experiment, DSC was used to determine the T_{g} onset of the PL; 0.1% ND-OH was compared directly with 0.1% nano-HA and unfilled polymer, all three groups underwent the same CM and SSPC processing. DSC ramps were set from 30° C. to 250° C. at 10° C./minute. Thermal history was not erased with pre-cycling because all of the samples came from precisely the same thermal history process SSPC annealing and PID controlled compression mold with 2 hour cooling cycle. Representative curves of the DSC results are shown in FIG. 26, in which the addition of HA reduces both the T_{g} onset temperature and the magnitude of heat exchanged. These results indicate that HA may decrease crystallinity and increases the polymer's free volume, making the HA composite sample undergo a phase transition at a lower temperature with less input energy. The composites containing 0.1% ND-OH show divergent results, in which both the T_{g} onset temperature increase and the magnitude of heat absorbed is much more to undergo phase transition; these results indicate stronger and more thermodynamically stable interactions between the ND and the PL.

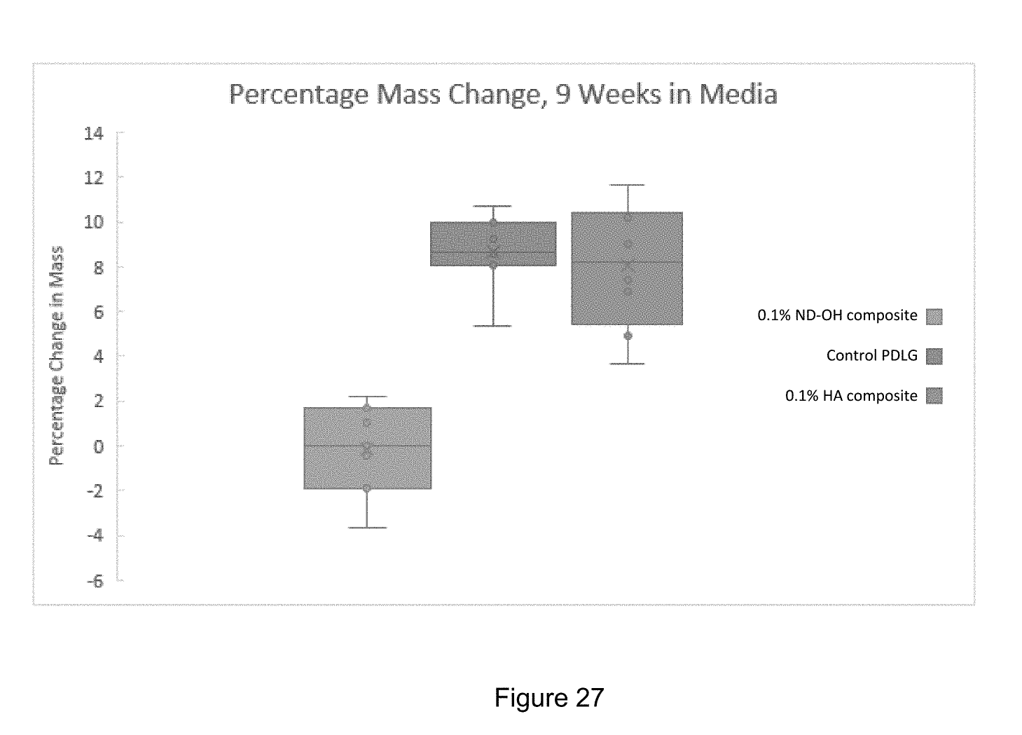

A 9-week degradation study was performed by submerging samples of various sizes in 50 mL of αMEM with 10% FBS and 1% Penn/Strep in 50 mL conical tubes. The 50 mL conical tubes were placed in a blotting oven with temperature set to 37° C. The mass of each sample was collected before the start of the test. At the termination, samples were washed 3× in DI water and dried under vacuum overnight for re-massing. The sample masses are plotted in FIG. 27. The 0.1% ND-OH composite (grey/left) demonstrated no weight gain, significantly different (p<0.001, n=8) from both the control/CM polymer (blue/center) and the 0.1% HA composite (orange/right).

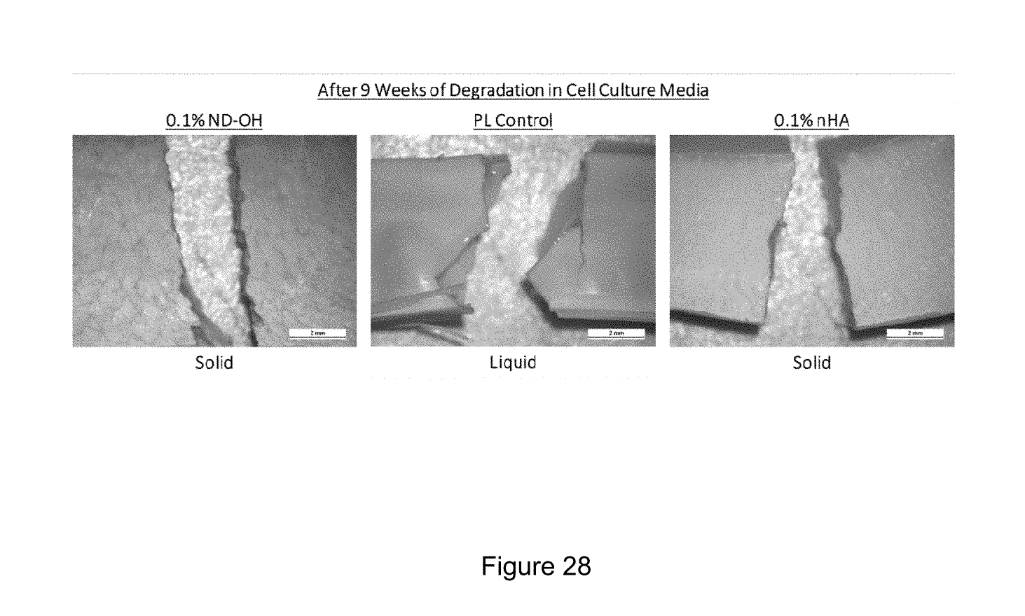

Even more peculiar is that the control (PL without nano-filler) turned into a complete liquid within a thin shell (FIG. 28). As DSC had revealed that the HA group should have required the least amount of input energy to free the polymer chains and accelerate bulk degradation, the presence of HA must have acted as a buffer to slow degradation. ND-OH composites also demonstrated the same solid attributes, but without the weight gain and decrease in T_{g} onset observed in the HA group, indicating that other mechanisms inhibited degradation (i.e. low free volume). The controls likely swelled, rapidly degraded internally due to locally acidic pH, but maintained a thin shell of PL that was actively buffered by the cell culture media.

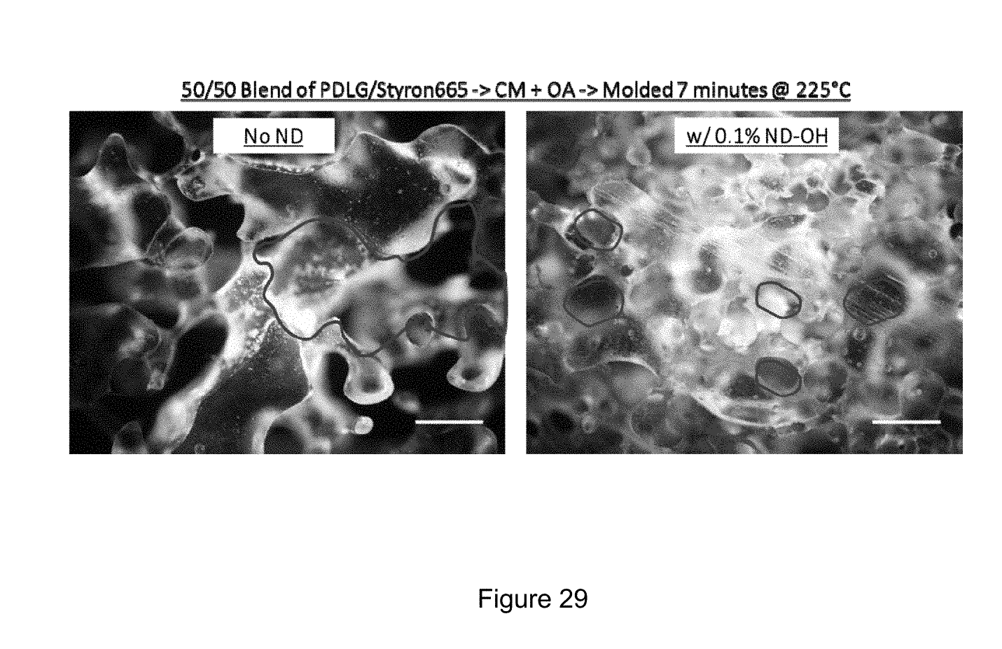

The next experiments have combined the use of 50/50 porous PL/PS blends with 0.1% ND-OH and the steps of CM and SSPC. As can be observed in FIG. 29, the coarsening of the pore size is slowed but does not decomposed into one phase dispersing another.

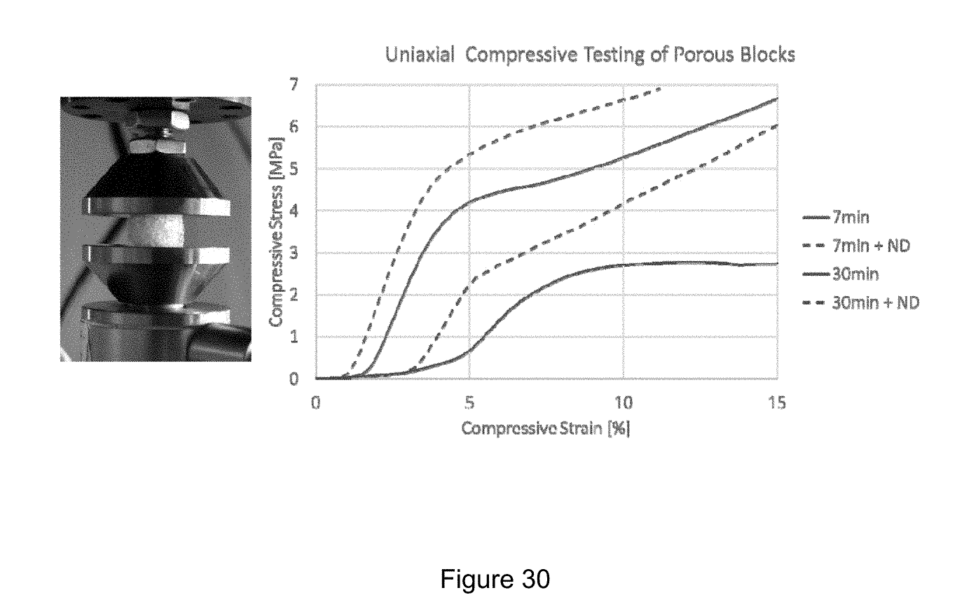

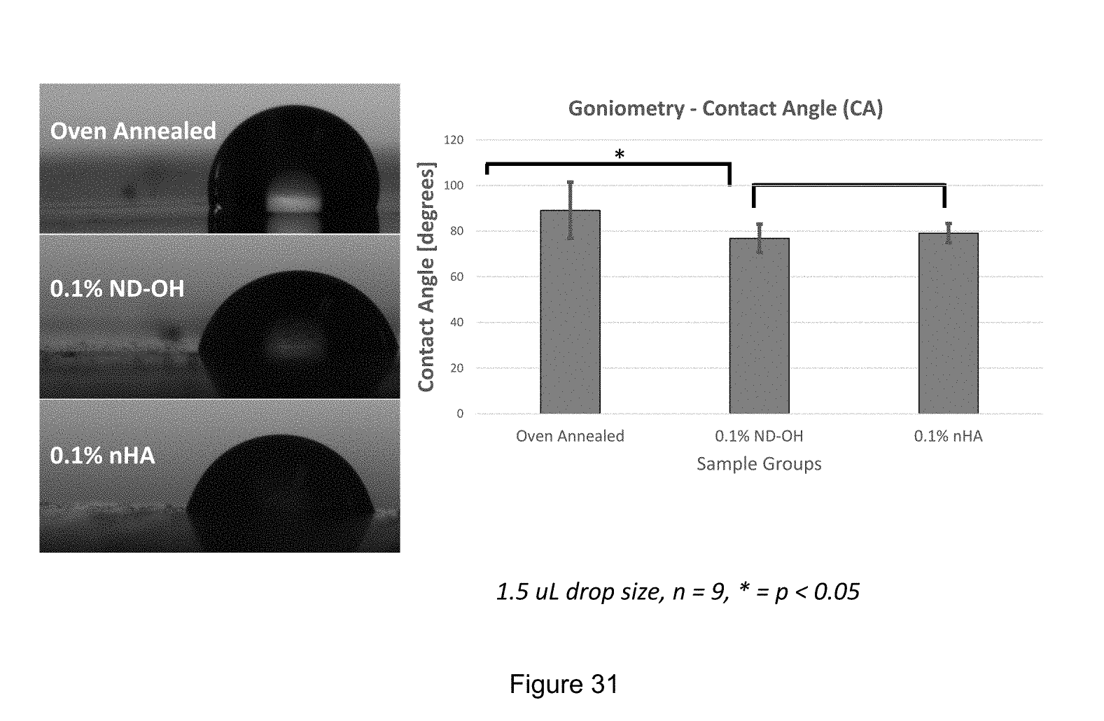

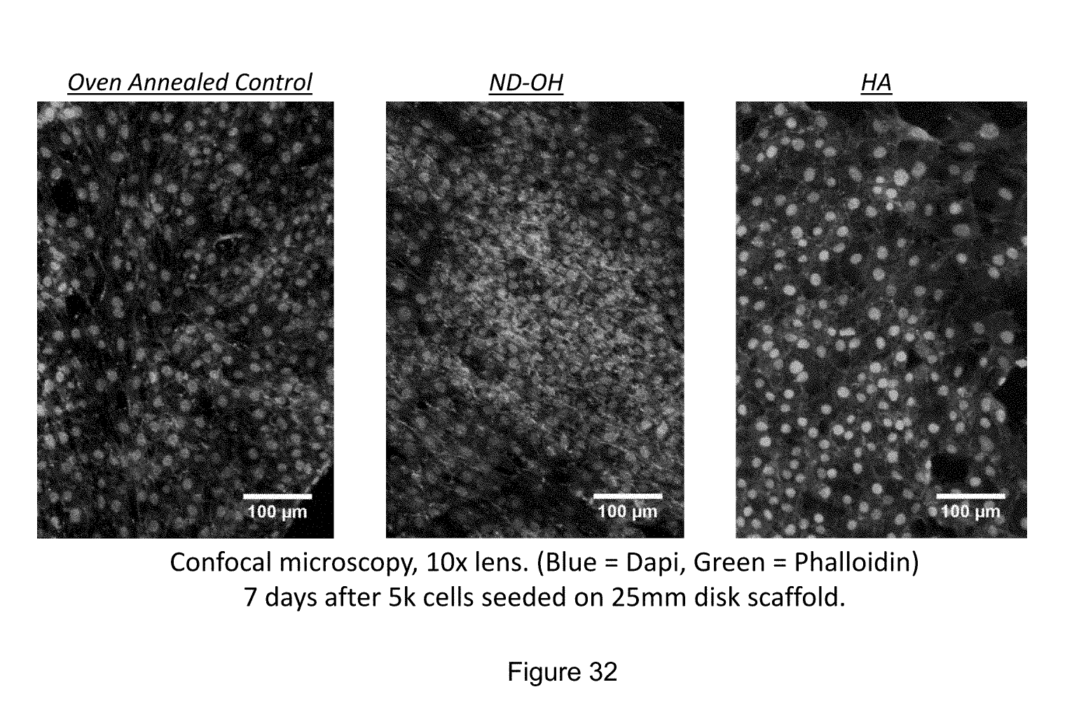

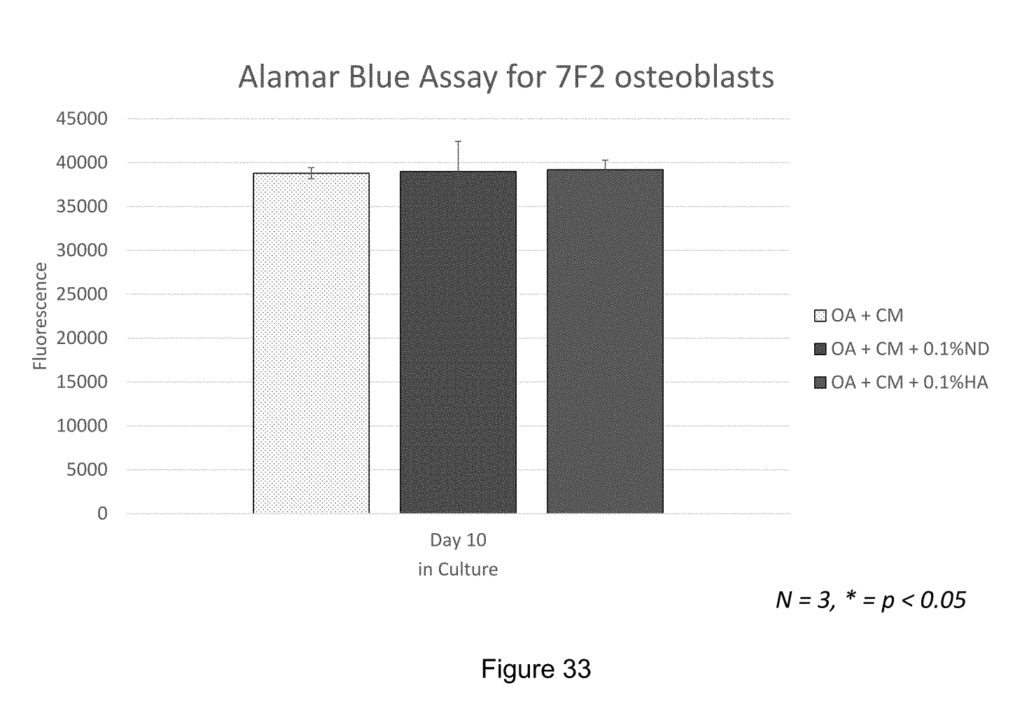

Under uniaxial compression testing of 50% porous blocks containing either 0.0% or 0.1% ND-OH, the presence of ND-OH improved all facets of mechanical integrity. Representative stress-strain curves are shown in FIG. 30.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of manufacturing a biomaterial comprising:
mixing a biocompatible polymer with a nanomaterial;
mechanically processing the polymer-nanomaterial mixture to disperse the nanomaterial throughout the mixture; and
vacuum annealing the mixture to promote covalent bond formation between the polymer and nanomaterial.

2. The method of claim 1, wherein mechanically processing the polymer-nanomaterial mixture comprises cyromilling of the mixture.

3. The method of claim 1, wherein mechanically processing the polymer-nanomaterial mixture comprises solid-state shear pulverization of the mixture.

4. The method of claim 1, wherein mechanically processing the polymer-nanomaterial mixture produces reactive polymer chain ends on the polymer.

5. The method of claim 1, wherein the biocompatible polymer is selected from the group consisting of polyglycolic acid (PGA), polylactic acid (PLA), poly-L-lactic acid (PLLA), poly-D/L-lactic acid with polyglycolic acid (PDLLA-co-PGA), poly-L-lactic acid-co-glycolic acid (PLGA), poly(D,L-Lactide-co-Glycolide) (PDLG), PDLLA, polydioxanone (PDS), poly(ϵ-caprolactone) (PCL), polycaprolactone (PCL) with alginate, polyhydroxybutyrate (PHB), polycarbonate (PC), N-vinyl pyrrolidone copolymers, polyorthoester, chitosan, poly(2-hydroxyethyl-methacrylate) (PHEMA), PEG (polyethylene glycol), and hyaluronic acid.

6. The method of claim 1, wherein the nanomaterial is selected from the group consisting of carbon nano-diamonds, detonation nano-diamonds, hydroxyapatite, tricalcium-phosphate, silica, bioglass, graphene oxides, single-walled carbon nanotubes, and multi-walled carbon nanotubes.

7. The method of claim 6, wherein the nanodiamonds are surface functionalized nanodiamonds.

8. The method of claim 7, wherein the surface functionalized nanodiamonds comprise one or more surface groups selected from the group consisting of: —OH, —COOH, and —NH2.

9. The method of claim 1, wherein vacuum annealing is conducted at a pressure of about 0.001 to 20 torr.

10. The method of claim 1, wherein vacuum annealing is conducted at or below the melting temperature of the biopolymer.

11. The method of claim 1, further comprising the step of compression molding the mixture to form a fixation device.

12. The method of claim 1, wherein the mixture further comprises a porogen and wherein the method further comprises removal of the porogen, thereby forming a porous fixation device.

13. The method of claim 11, wherein the fixation device is selected from the group consisting of a screw, pin, rod, plate, and staple.

14. A biomaterial comprising a polymer matrix comprising one or more biocompatible polymers and a nanomaterial, wherein the nanomaterial is covalently bonded to polymer matrix.

15. The biomaterial of claim 14, wherein the biocompatible polymer is selected from the group consisting of polyglycolic acid (PGA), polylactic acid (PLA), poly-L-lactic acid (PLLA), poly-D/L-lactic acid with polyglycolic acid (PDLLA-co-PGA), poly-L-lactic acid-co-glycolic acid (PLGA), poly(D,L-Lactide-co-Glycolide) (PDLG), PDLLA, polydioxanone (PDS), poly(ϵ-caprolactone) (PCL), polycaprolactone (PCL) with alginate, polyhydroxybutyrate (PHB), polycarbonate (PC), N-vinyl pyrrolidone copolymers, polyorthoester, chitosan, poly(2-hydroxyethyl-methacrylate) (PHEMA), PEG (polyethylene glycol), and hyaluronic acid.

16. The biomaterial of claim 14, wherein the nanomaterial is selected from the group consisting of carbon nano-diamonds, detonation nano-diamonds, hydroxyapatite, tricalcium-phosphate, silica, bioglass, graphene oxides, single-walled carbon nanotubes, and multi-walled carbon nanotubes.

17. The biomaterial of claim 14, wherein the nanodiamonds are surface functionalized nanodiamonds.

18. The biomaterial of claim 14, wherein the surface functionalized nanodiamonds comprise one or more surface groups selected from the group consisting of: —OH, —COOH, and —NH2.

19. (canceled)

20. The biomaterial of claim 14, wherein the biomaterial is an orthopedic fixation device.

21. The biomaterial of claim 20, wherein the orthopedic fixation device is selected from the group consisting of a screw, pin, rod, plate, and staple.

22. (canceled)
